(19) United States
(12) Patent Application Publication
(10) Pub. No.: US 2025/0018181 A1
(43) Pub. Date: Jan. 16, 2025

(54) PIEZOELECTRIC SPINAL IMPLANT ASSEMBLY HAVING ENDPLATES WITH NON-PLANAR SURFACES

(71) Applicants: Ziev Moses, Needham, MA (US); Kevin Chappuis, Saugus, MA (US); Luke Diehl, Medford, MA (US); Lance Smith, Edmond, OK (US)

(72) Inventors: Ziev Moses, Needham, MA (US); Kevin Chappuis, Saugus, MA (US); Luke Diehl, Medford, MA (US); Lance Smith, Edmond, OK (US)

(21) Appl. No.: 18/900,021

(22) Filed: Sep. 27, 2024

Related U.S. Application Data
(63) Continuation-in-part of application No. 18/308,406, filed on Apr. 27, 2023.
(63) Continuation-in-part of application No. 18/308,455, filed on Apr. 27, 2023.
(60) Provisional application No. 63/335,343, filed on Apr. 27, 2022.
(60) Provisional application No. 63/396,019, filed on Aug. 8, 2022.
(60) Provisional application No. 63/335,343, filed on Apr. 27, 2022.

(51) Int. Cl. A61N 1/32; A61F 2/30; A61F 2/44

(52) U.S. Cl. CPC A61N 1/326; A61F 2/4455; A61F 2002/30087

(57) ABSTRACT

A spinal implant assembly, a kit for forming same and a method for forming same. The spinal implant assembly comprises: a first endplate having a first non-planar inner surface; a second endplate having a second non-planar inner surface facing toward the first non-planar inner surface; at least one fastener attaching the first endplate and the second endplate to one another; and a layer including a piezoelectric material (piezoelectric layer), the piezoelectric layer between the first non-planar inner surface and the second non-planar inner surface and having a non-planar upper surface facing toward the first non-planar inner surface and a non-planar lower surface facing toward the second non-planar inner surface, wherein, at a front region of the spinal implant assembly, the first non-planar inner surface and the second non-planar inner surface define a vertically biased interface.

### CROSS-REFERENCE TO RELATED APPLICATIONS

This application is a continuation-in-part of U.S. Utility patent application Ser. No. 18/308,406 entitled “PIEZOELECTRIC SPINAL IMPLANT AND METHODS OF MAKING AND USING SAME” filed on Apr. 27, 2023, which claims priority to U.S. Provisional Patent Application Ser. No. 63/335,343 entitled “PIEZOELECTRIC SPINAL IMPLANT AND METHODS OF MAKING AND USING SAME” filed on Apr. 27, 2022, both of which are incorporated herein by reference in their entireties. This application is further a continuation-in-part of U.S. Utility patent application Ser. No. 18/308,455 entitled “PIEZOELECTRIC ORTHOPEDIC IMPLANT AND METHODOLOGY” filed on Apr. 27, 2023, which claims priority to two U.S. Provisional Patent Applications, namely, 63/335,343 entitled “PIEZOELECTRIC SPINAL IMPLANT AND METHODS OF MAKING AND USING SAME” filed on Apr. 27, 2022, and U.S. Provisional Patent Application Ser. No. 63/396,019 entitled “PIEZOELECTRIC ORTHOPEDIC IMPLANT AND METHODOLOGY,” and filed on Aug. 8, 2022, all three of which are also incorporated herein by reference in their entireties.

### FIELD

The present application relates to spinal implants comprising piezoelectric material capable of stimulating bone and tissue growth around the implant.

### BACKGROUND

Electrical stimulation has long been used to stimulate bone growth and promote fusion after spinal implant surgery. If, after a period of recovery, a patient does not naturally generate the type of growth anticipated by their physician, a second surgery may be required to place an electrical implant and associated battery under the skin, with a cathode running to the area of fusion around the bone. This type of treatment is generally only used as a secondary means of stimulating bone growth given its invasive nature and need for additional surgery.

Alternatively, patients may be instructed to wear a noninvasive vest or harness or to apply small skin pads/electrodes that are capable of generating a weak electrical current within the target sight using either pulsed electromagnetic fields, capacitive coupling, or combined magnetic fields. However, there is limited data evidencing the effectiveness of this type of noninvasive treatment. In addition contemporary piezoelectric spinal implants comprise a biocompatible frame or cage structure that incorporates one or more piezoelectric components. These components are positioned to maximize their exposure to the compressive and shear forces that occur during spinal movement. The electrical output generated by these piezoelectric elements is believed to stimulate osteogenesis and accelerate the fusion process.

### BRIEF DESCRIPTION OF THE FIGURES

Further embodiments of the present application can be understood with reference to the appended figures.

FIG. 1A is a side perspective view of a spinal implant assembly according to an embodiment.

FIG. 1B is a perspective view of a spinal implant assembly and inserter combination according to an embodiment.

FIGS. 1C and 1D are side perspective views of a spinal implant assembly according to an embodiment while the assembly is undergoing shear and compressive loading.

FIG. 2 is a top perspective view of top and bottom endplates of an embodiment of a spinal implant assembly.

FIGS. 3A-3C show a side-elevational view, a bottom view and a top view of the top endplate of FIG. 2.

FIGS. 4A-4C show a side-elevational view, a bottom view and a top view of the bottom endplate of FIG. 2.

FIGS. 5A-5C show respective perspective exploded views of a top endplate, a bottom endplate and a piezoelectric layer for a spinal implant assembly according to another embodiment.

FIGS. 6A-6C show a spinal implant assembly according to yet another embodiment, where screws are made of an electrically insulating material.

FIGS. 7A and 7B show a side-elevational view and a top view of a spinal implant assembly according to a further embodiment including screws and insulating rings.

FIGS. 8A-8C show a side-elevational, partially exploded view, a side perspective partially exploded view, and a front elevational partially perspective cross-sectional view of a spinal implant assembly according to some other embodiments using screws and insulating rings.

FIG. 9 is a perspective view of a lag screw and insulating ring combination according to an embodiment.

FIG. 10 is a side perspective partially transparent view of a spinal implant assembly according to yet another embodiment involving screws and insulating rings.

FIG. 11 is a perspective view of screws and insulating rings used in FIG. 10.

FIGS. 12A-12D show, respectively, a perspective side-elevational view of a spinal implant assembly according to an embodiment involving the use of brackets, and three respective stages of assembly of the spinal implant assembly in perspective view.

FIGS. 13A and 13B show, respectively, a side perspective partially transparent view of a spinal implant assembly and a top view of the spinal implant assembly according to yet another embodiment involving the use of brackets.

FIG. 14 shows a perspective view of a bracket of the embodiment of FIGS. 13A-13B.

FIGS. 15A, 15D and 15E show, respectively, a perspective partially transparent view, a perspective exploded view and a side-elevational view of a spinal implant assembly according to an embodiment involving the use of struts.

FIGS. 15B and 15C show, respectively, a top perspective view and a side-elevational view of a bottom endplate of the spinal implant assembly of the embodiment of FIGS. 15A, 15D and 15E.

FIG. 16 shows two embodiments of screw heads for use in orthopedic implant assemblies.

FIG. 17 shows a spinal cord including a spinal implant assembly according to some embodiments having been implanted therein.

### DETAILED DESCRIPTION

Spinal implants that incorporate piezoelectric components aim to address the critical need for improved bone growth stimulation and fusion outcomes in spinal surgery patients. Piezoelectric materials, known for their ability to generate an electric charge in response to mechanical stress, have shown promise in enhancing the biological response to implants and promoting faster, more robust healing. Traditionally, spinal fusion procedures have relied on passive implants that provide structural support but offer limited active contribution to the healing process. The integration of piezoelectric elements into spinal implants has represented a paradigm shift in this field, as it allows for the generation of localized electrical stimulation in response to the natural mechanical forces experienced by the spine during daily activities.

In accordance with one embodiment, a spinal implant assembly capable of generating an electric output includes: a first endplate having a first non-planar inner surface; a second endplate having a second non-planar inner surface facing toward the first non-planar inner surface; at least one fastener attaching the first endplate and the second endplate to one another; and a layer including a piezoelectric material (piezoelectric layer), the piezoelectric layer between the first non-planar inner surface and the second non-planar inner surface and having a non-planar upper surface facing toward the first non-planar inner surface and a non-planar lower surface facing toward the second non-planar inner surface, wherein, at a front region of the spinal implant assembly, the first non-planar inner surface and the second non-planar inner surface define a vertically biased interface.

Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Various embodiments containing different features and aspects are shown in this disclosure. It should be understood that other embodiments containing some of these features and aspects, and other features and aspects known in the art, are within the scope of this disclosure, even if such an exact combination is not shown or described specifically. Also, it is to be understood that the phraseology and terminology employed herein is for the purpose of description and should not be regarded as limiting.

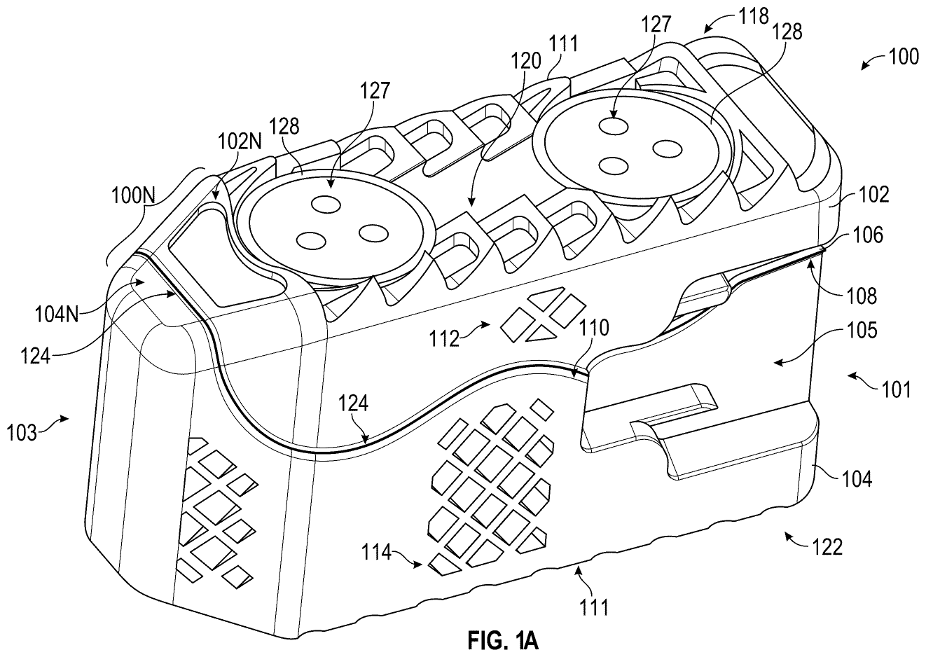

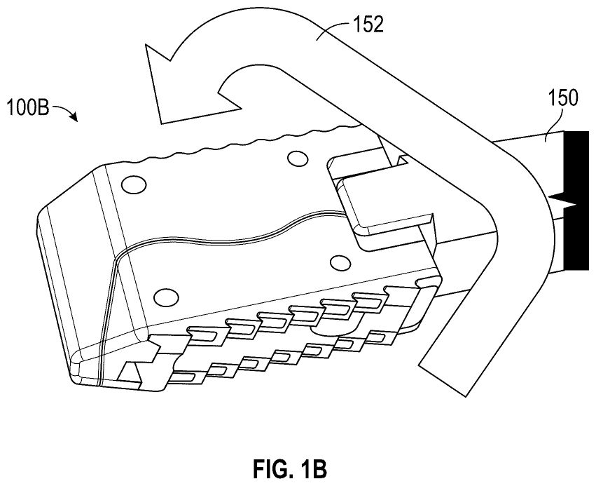

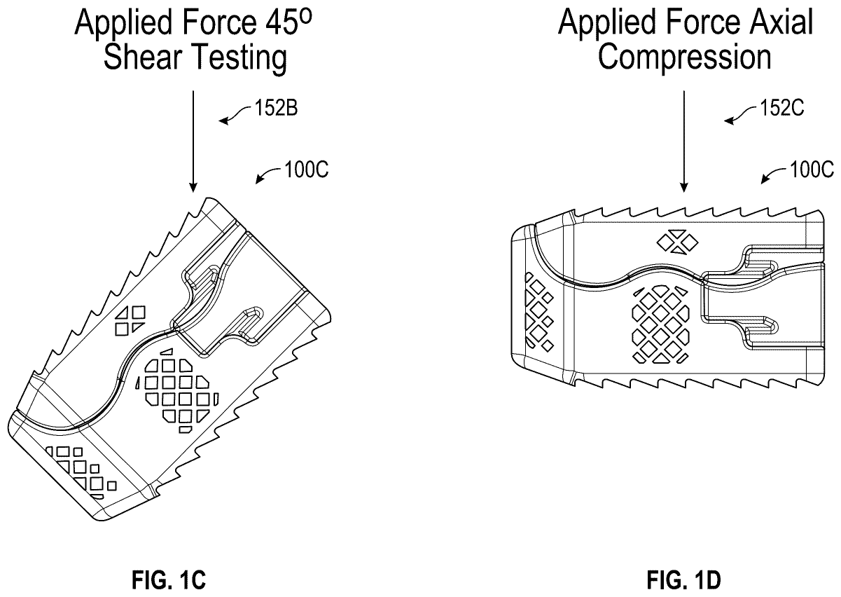

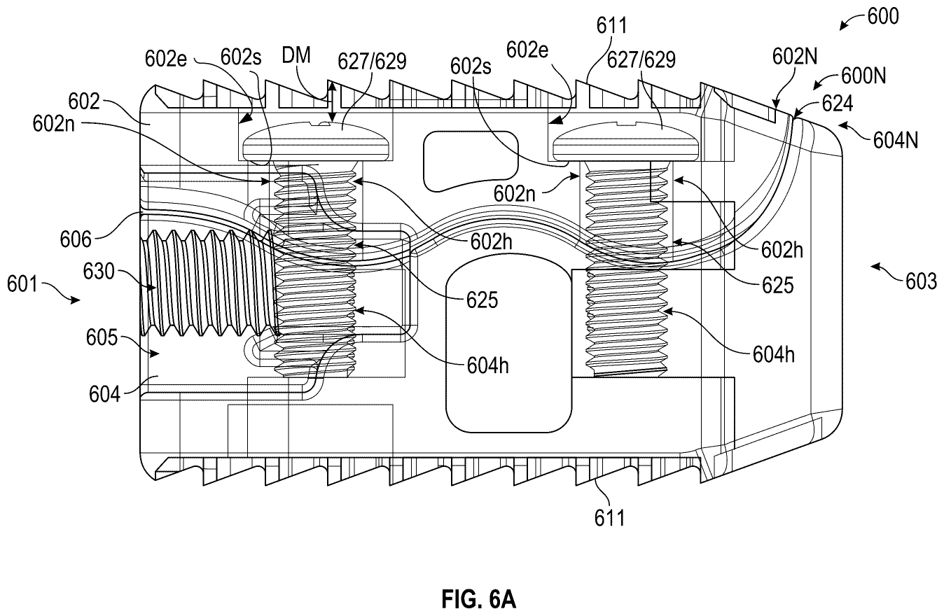

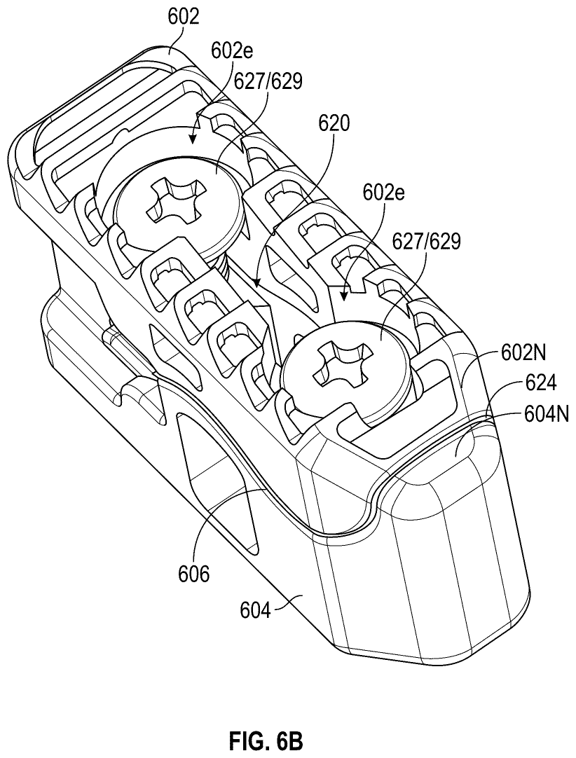

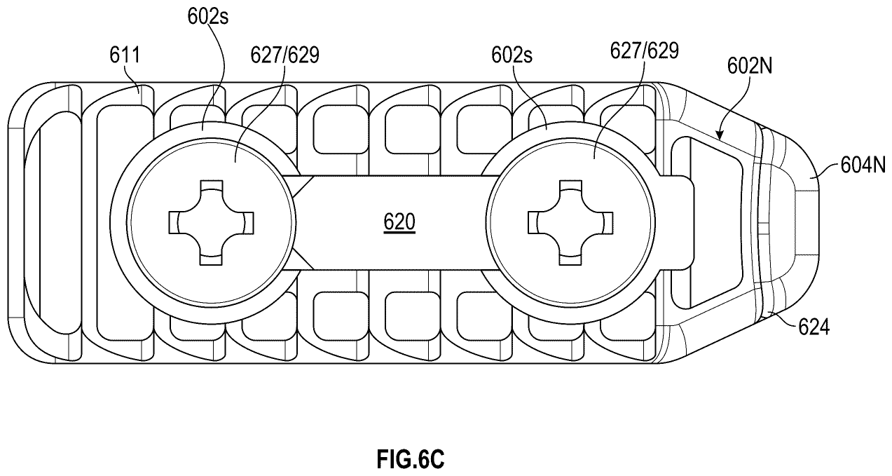

Figures described herein, except for FIGS. 9, 11 and 14, show various embodiments of a dual endplate spinal implant assembly having a layer including a piezoelectric (hereinafter “piezoelectric layer”) material disposed between, conforming to and adjacent a top endplate of the spinal implant assembly and a bottom endplate of the spinal implant assembly. For ease of description, let us first refer to FIGS. 1A-1B, 2, 3A-3C, 4A-4C, and 5A-5C in the context of a description of features of the top endplate, the bottom endplate, and the piezoelectric layer of a spinal implant assembly that could be common across some embodiments, such as, for example, across the embodiments of FIGS. 6A-6C, 7A-7B, 8A-8B, 10, 12A-12D, 13A-13B, 15A-15E. For the latter reason, like features as between FIGS. 1, 2, 3A-3C, 4A-4C, and 5A-5C will be referred to herein with like reference numerals, and will not be described in detail every time the description moves from one embodiment to another.

A piezoelectric layer as referred to herein may include one or more piezoelectric materials, such as, for example, a polymer exhibiting piezoelectric characteristics. For example, a piezoelectric material as referred to herein may include a piezoelectric polymer, such as, by way of example, polyvinylidene difluoride (PVDF). A piezoelectric layer as referred to herein may include one or more layers, may include one or more non-piezoelectric materials in addition to at least one piezoelectric material, and it may have a uniform or non-uniform material composition throughout.

Referring first to FIG. 1A, an embodiment of a spinal implant assembly 100 is shown that includes a top endplate 102, a bottom endplate 104, and a piezoelectric layer 106 therebetween. Piezoelectric layer 106 is disposed between, conforms to and is adjacent a non-planar lower surface 108 of top endplate 102 at an upper surface thereof, and a non-planar inner surface 110 of bottom endplate 104 of the spinal implant assembly 100. Non-planar inner surface 108 of the top endplate conforms to the non-planar inner surface 110 of the bottom endplate.

The top endplate 102 and the bottom endplate 104 preferably are unitary, that is, they are preferably make of a single piece of material, for example fabricated by way of 3D printing, or any other suitable technique. Making the top endplate 102 and the bottom endplate 104 unitary among other things imparts mechanical robustness to a spinal implant assembly resulting therefrom, which becomes important especially during insertion of the spinal implant assembly in between vertebrae, for example, during rotation thereof into an upright position according to one example manner of inserting the implant assembly, as will be described in further detail in relation to FIG. 1B.

The top endplate 102 and the bottom endplate 104 may be made of a same material, or they may be made of different materials as compared with one another. Any one of the top endplate 102 and the bottom endplate 104 may include an electrically conductive material such as, for example, one or more of titanium, tungsten, stainless steel, platinum or gold, it being understood that the latter list of materials is to include alloys of the same.

By “non-planar” in the context of a surface, what is meant herein is a surface that is not flat, such as a surface with at least one of undulations/waves, zig-zags, curves, bends, corners, or creases, oriented in any direction. The surfaces may be smooth, or they may contain any number of sharp features.

In the context of embodiments, a non-planar interface with a piezoelectric layer is to distribute compressive or shear forces applied to the endplates unevenly across the piezoelectric layer.

In order to arrive at embodiment, through experimentation, inventors have discovered that, a same axial/compressive force, a same shear force, and a same force combining the two, when applied to two endplates of a spinal implant assembly with a piezoelectric layer therebetween, result in the generation of a higher voltage output across the piezoelectric layer when the piezoelectric layer defines, in the assembly, 2D (2-dimensional) longitudinally extending undulations/wave patterns as opposed to planar inner surfaces or to inner surfaces with a single wave. In addition, inventors have discovered that, a same axial/compressive force, a same shear force, and a same force combining the two, when applied to two endplates of a spinal implant assembly with a piezoelectric layer therebetween, result in the generation of a higher voltage output across the piezoelectric layer when the piezoelectric layer defines, at respective inner surfaces thereof, 3D (3-dimensional) undulations/wave patterns (i.e., extending both longitudinally and both transversely to the longitudinal direction and horizontally) as opposed to planar inner surfaces or to inner surfaces with a single wave. Inventors has discovered that such increased voltage outputs advantageously result in associated improved (e.g., faster and more copious) osteogenesis within the body. A spinal implant assembly according to embodiments corresponds to a spinal interbody implant assembly featuring piezoelectric properties as a first electroactive spinal implant capable of providing electrical stimulation directly to a load-bearing disc space (where spinal fusion primarily occurs) in a spinal column without the need for batteries, wires, or circuits. Advantageously, a non-planar configuration of the piezoelectric layer when it is assembled into a spinal implant assembly according to embodiments ultimately results in improved osteogenesis as compared with spinal implant assemblies of the prior art where the piezoelectric layer is flat.

For example, pre-clinical research has demonstrated a significant increase in osteogenesis in sheep spine using a spinal implant assembly according to some embodiments where the piezoelectric layer is non-planar with one or multiple waves, as opposed to spinal implants where the piezoelectric layer is flat/planar.

By a non-planar surface A “conforming to” another non-planar surface B, what is meant herein is that surface A and surface B exhibit inter-fitting surfaces, enabling a complementary nesting configuration of surfaces A and B.

In the context of embodiments, non-planar surfaces 108 and 110 are configured such that, when subjected to compressive and/or shear forces, they apply compressive and/or shear forces to piezoelectric layer 106 in an unevenly distributed manner across the piezoelectric layer. Piezoelectric layer 106 corresponds to a non-planar boundary or “interface” or “seam” 124 between the top endplate 102 and the bottom endplate 104.

In the context of the instant description, the words “interface” or “seam” or “boundary” between A and B do not necessarily mean that A and B must be directly physically touching/adjacent, although they could. Rather, these words pertain to an interaction between surfaces of A and B, whether direct or indirect.

The top endplate 102 and the bottom endplate 104 may include, at surfaces thereof, for example at side surfaces thereof, lattice structures 112 (for the top endplate 102) and 114 (for the bottom endplate 104) that open into a lumen 120 of the spinal implant assembly 100. The lattice structures can have a uniform stiffness throughout the implant assembly, or a variable stiffness that is variable based on location. Although the embodiment of FIG. 1A shows lattice structures defining, generally, diamond-shaped openings, embodiments are not so limited, and include any configuration of lattice structures anywhere on the top endplate or on the bottom endplate as would be recognized by a person of skilled in the art.

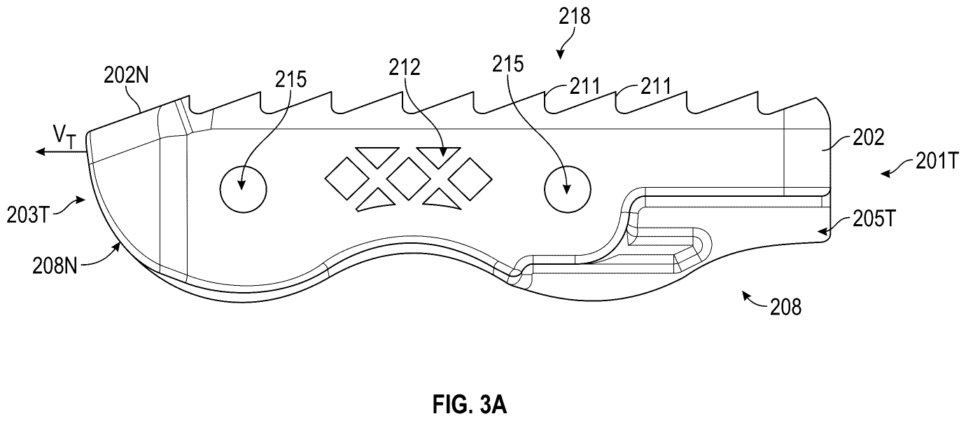

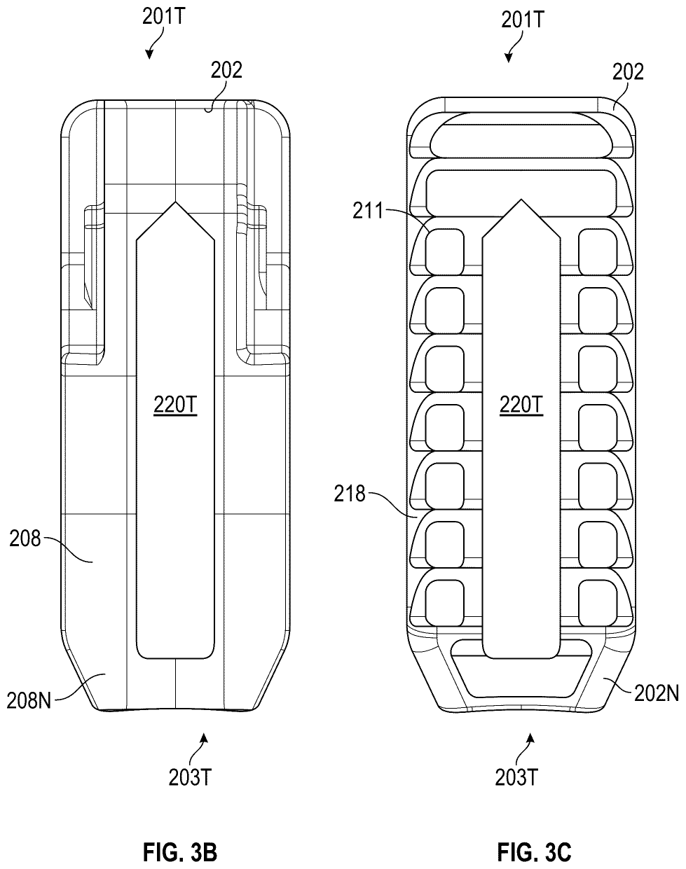

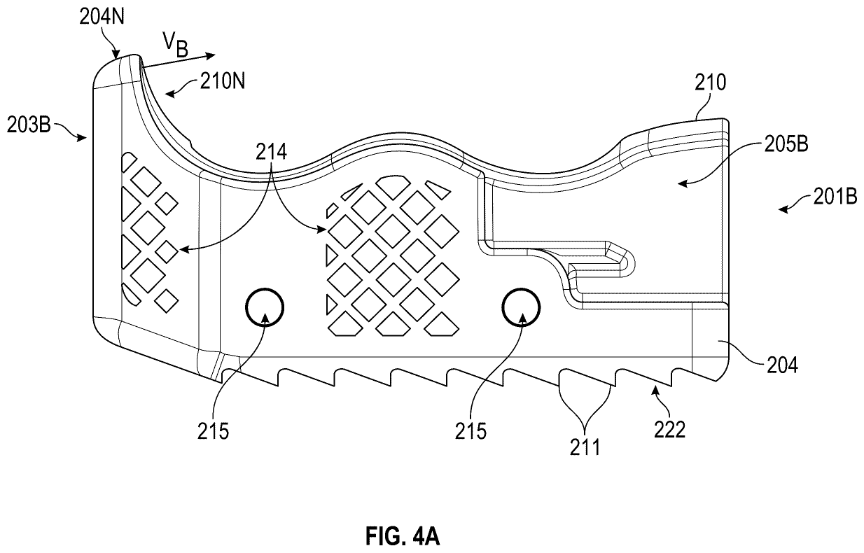

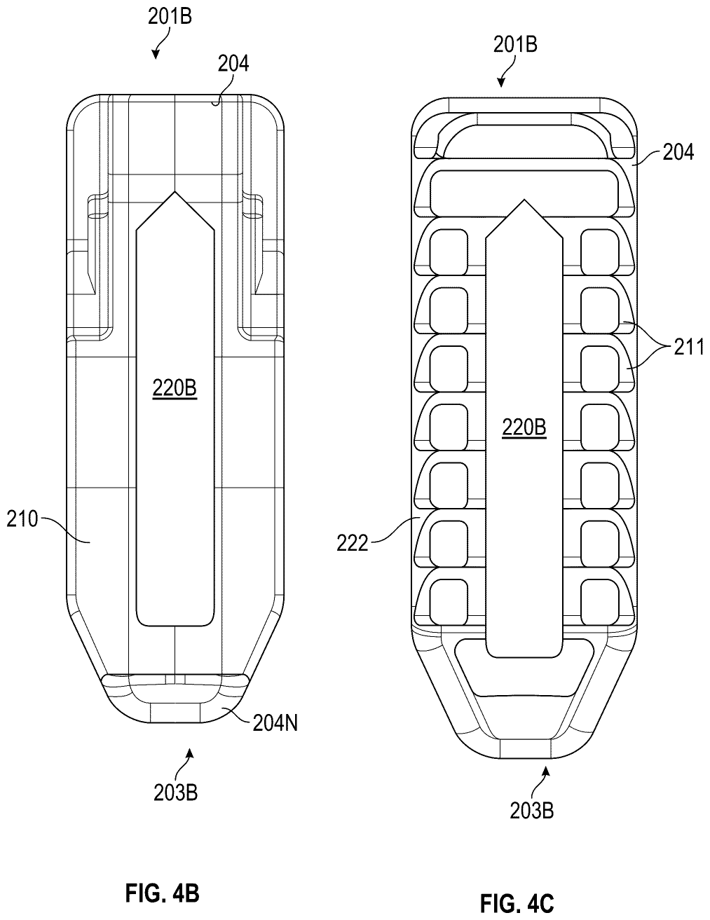

Lumen 120 is defined by way of a registration of a corresponding top lumen segment of the top endplate with a corresponding bottom lumen segment of the bottom endplate (as best seen in FIGS. 3A-3C and FIGS. 4A-4C, to be described further below).

The top endplate 102 has a top surface 118 and the bottom endplate has a bottom surface 122, where top surface 118 and bottom surface 122 represent respective outer-facing surfaces of the top and bottom endplates that are to be positioned adjacent to respective vertebrae when a spinal implant assembly including such top and bottom endplates is inserted into the spine of a patient. Surfaces 118 and 122, as seen in FIG. 1A, may include protrusions in the form of teeth 111 thereon as shown. Although teeth are shown in FIG. 1A, embodiments envisage other types of protrusions, such as, for example, studs, flanges, spikes or fins.

In the shown embodiment of FIG. 1A, the spinal implant assembly 100 includes a back region 101, and an opposing front region 103 longitudinally spaced from back region 101. The back region 101 includes a grooved recess 105 therein for receiving the head of a spinal implant inserter tool (not shown). The front region 103 includes an downwardly sloped and horizontally extending nose surface 100N of the spinal implant assembly 100. The downwardly sloped and horizontally extending nose surface 100N corresponds to a combination of downwardly sloped and horizontally extending nose surface 102N of top endplate 102 with a downwardly sloped and horizontally extending nose surface 104N of bottom endplate 104, where the combination is segmented by an upward facing portion of seam 124.

The components of spinal implant assembly, including the top endplate, the bottom endplate and the piezoelectric layer, may be assembled with one another using fasteners.

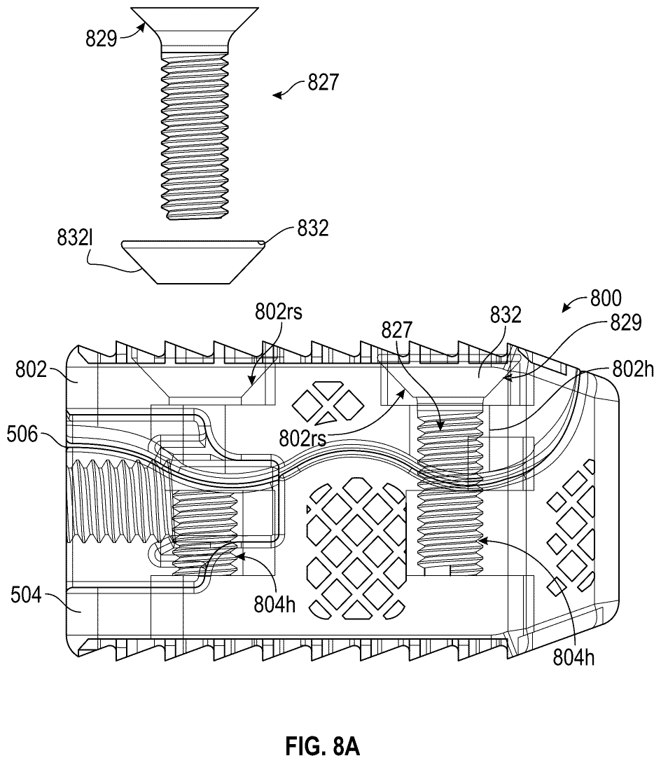

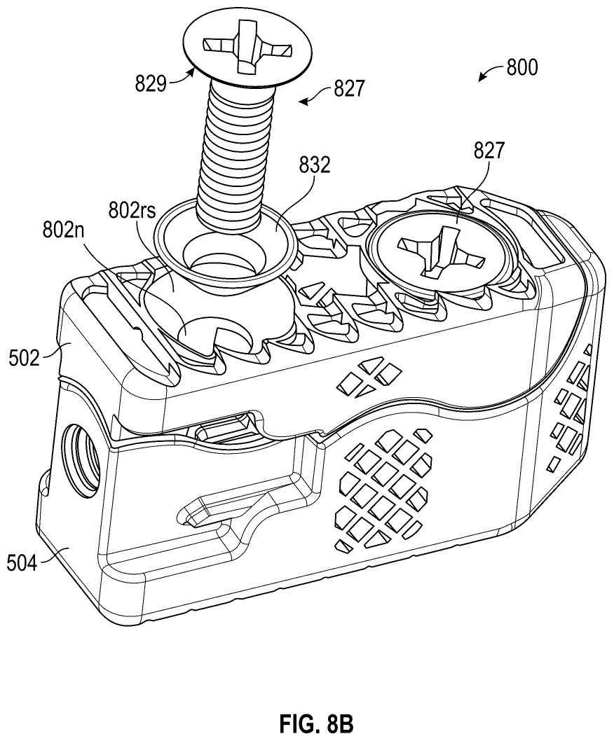

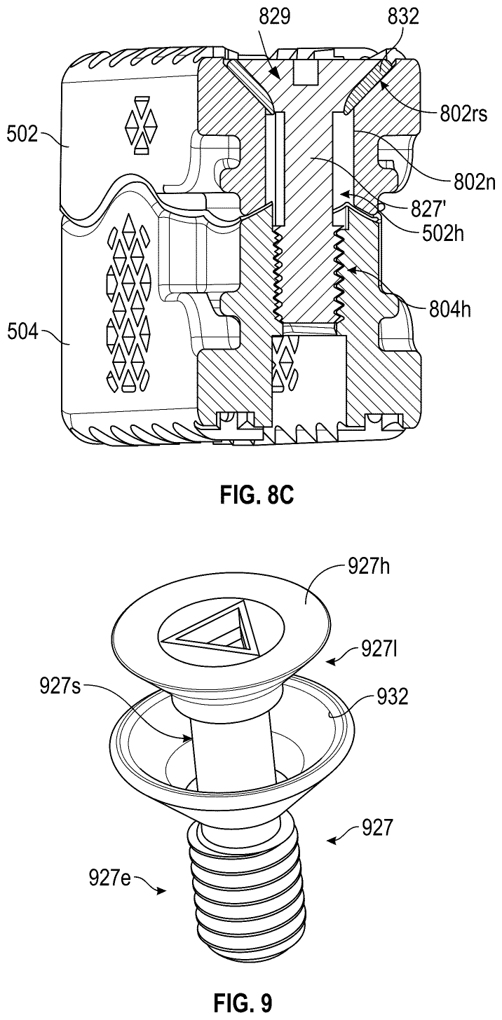

In the shown embodiment of FIG. 1A, the fasteners include a pair of screws 127 electrically insulated from the top endplate 102 by way of electrically insulating rings or washers 128, as will be explained in further detail below in relation to the embodiment of FIGS. 8A-8C.

Referring now to FIG. 1B, a perspective view is shown of a spinal implant assembly 100B attached to an inserter 150 according to an embodiment. FIG. 1B shows an arrow 152 suggesting a direction of rotation of a spinal implant assembly after insertion between two vertebrae. As suggested in FIG. 1B, according to an example embodiment, a spinal implant assembly may first be inserted between two vertebrae on its side, and thereafter rotated, using the inserter, into an upright position as suggested by curved arrow 152. As can be appreciated from FIG. 1B, insertion of a spinal implant assembly can subject the implant assembly to significant torsional stresses that can in some instances lead to a breakage of the spinal implant, especially where the implant includes components that are fastened/attached together, as is the case with embodiments set forth herein. According to another embodiment for inserting a spinal implant assembly as disclosed herein, such spinal implant assembly may be inserted already in an upright position, thus not requiring rotation. Still, even in such a scenario, the implant assembly would be subjected to substantial mechanical stresses, requiring for the same to be exhibit mechanical strength and structural integrity to be able to withstand the same.

Reference is now made to FIGS. 1C and 1D, which show a side perspective view of a spinal implant assembly 100C according to some embodiments, along with arrows 152B and 152C that suggest the direction of, respectively, an applied shear force to the spinal implant assembly at an angle of about 45 degrees with respect to a normal axis of an upper surface of the implant assembly, and an applied compression/axial force at an angle of zero degree with respect to such normal axis. Such shear and compression forces are to be applied in order for spinal implants to have more reliable strength during implantation and when subjected to multiple compression and shear forces after implantation.

An advantage of embodiments is that it provides a spinal implant assembly that exhibits reliable structural strength during insertion and throughout the course of its implanted lifetime within a spine.

Another advantage of embodiments is that it provides a multi-component spinal implant assembly assembled using reliable and robust fasteners in such a way as to ensure reliable placement of the spinal implant assembly and sturdiness of the same after implantation while at the same time ensuring electrical isolation of a top endplate of the spinal implant assembly from a bottom endplate thereof in order to preserve opposing polarity as between an upper surface and a lower surface of a piezoelectric layer between the two endplates where those endplates are electrically conductive.

Advantageously, embodiments of a spinal implant assembly as disclosed herein prevent short circuiting or offsetting positive/negative electrical output from the spinal implant assembly.

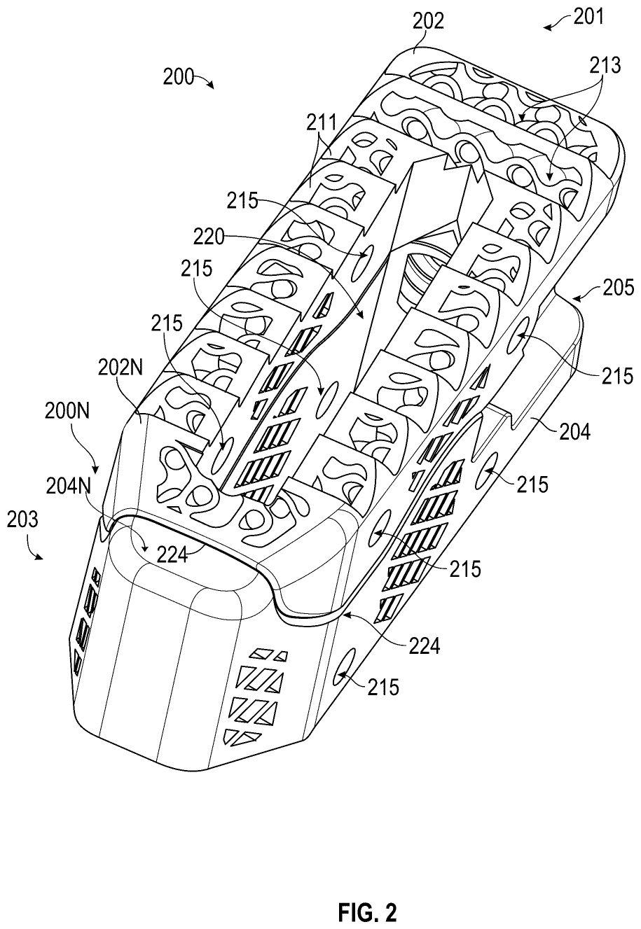

Reference will now be made to FIGS. 2, 3A-3C, 4A-4C, and 5A-5C, in which like components as compared with FIG. 1A will be referred to with like reference numerals. Unless specified otherwise, any description of such like components in the context of FIG. 1A may apply to such corresponding components in FIGS. 2, 3A-3C, 4A-4C, and 5A-5C below.

Referring now to FIG. 2, a top perspective view is shown of top and bottom endplates according to another embodiment, where the top and bottom endplates are shown in a nested/mated state. In FIG. 2, a piezoelectric layer and fasteners have been omitted for ease of description.

FIGS. 3A-3C respectively show a side-elevational view, a bottom plan view and a top plan view of top endplate 202 of FIG. 2.

FIGS. 4A-4C respectively show a side-elevational view, a bottom plan view and a top plan view of top endplate 202 of FIG. 2.

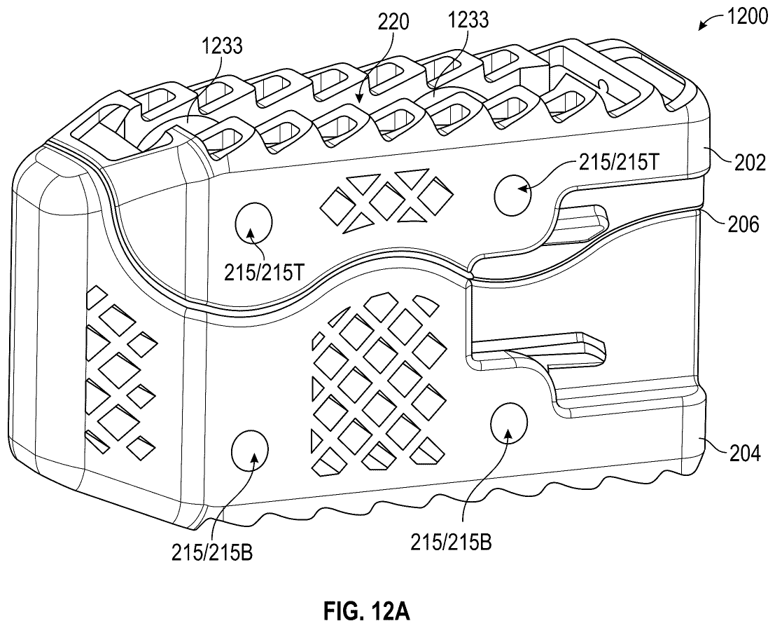

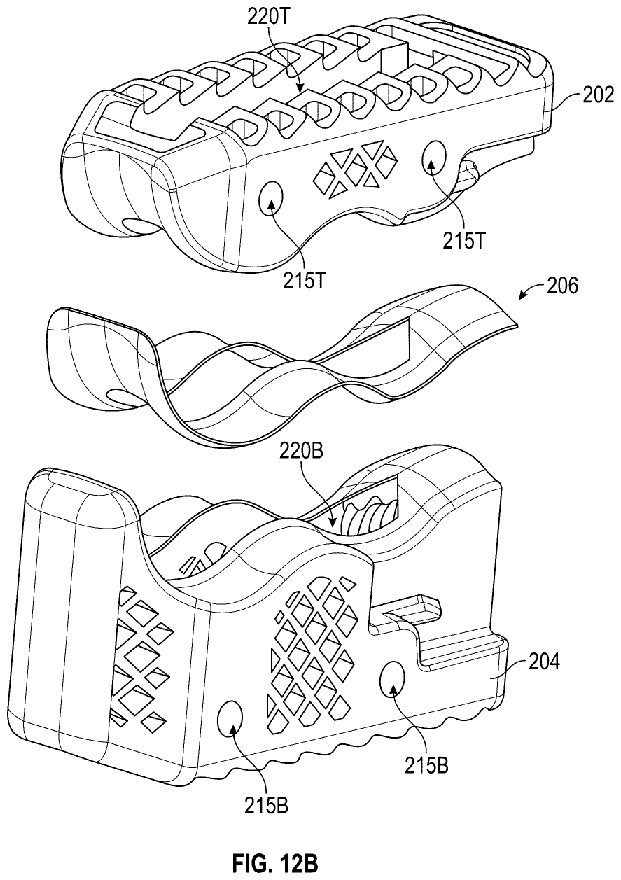

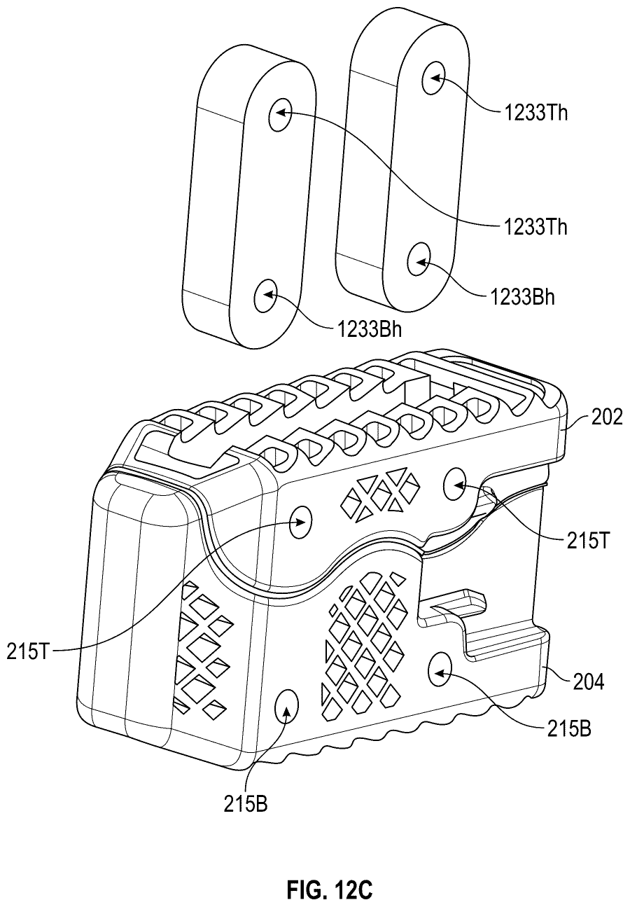

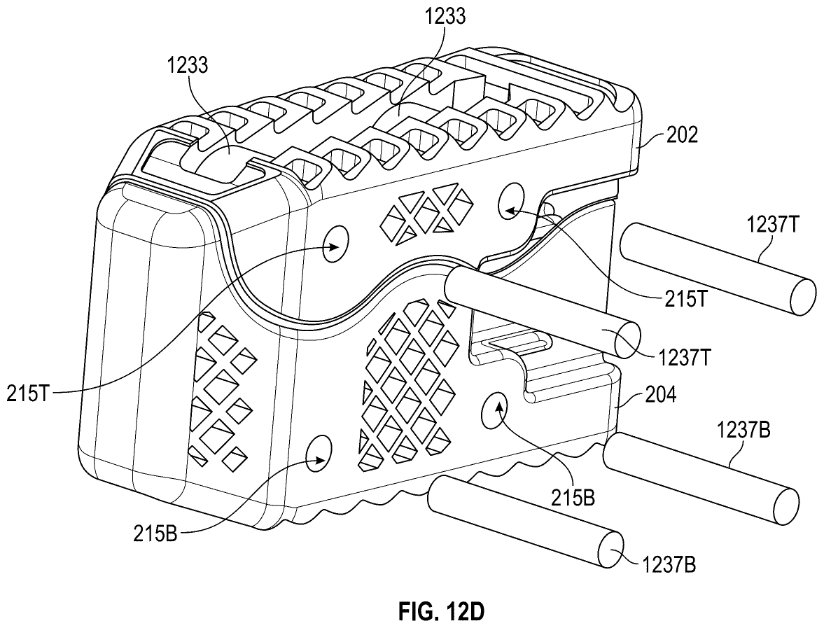

The top and bottom endplates of FIGS. 2, 3A-3C and 4A-4C may for example be used in embodiments depicted herein and described in further detail below in relation to FIGS. 12A-14.

FIG. 2 shows an assembly 200 including top endplate 202 and bottom endplate 204 each having inner surfaces that conform to one another to define a seam 224 as shown (noting that the scam 224 of FIG. 2 is not depicted as including a piezoelectric layer, for case of description only. Lumen 220 is defined by way of a registration of a corresponding top lumen segment of the top endplate with a corresponding bottom lumen segment of the bottom endplate. Outer-facing surfaces of top and bottom endplates include protrusions in the form of teeth 211 thereon as shown. Back region 201 includes a grooved recess 205 therein for receiving the head of a spinal implant inserter tool (not shown). The front region 203 includes an downwardly sloped and horizontally extending nose surface 200N of the spinal implant assembly 200. The downwardly sloped and horizontally extending nose surface 200N corresponds to a nose surface that is a combination of downwardly sloped and horizontally extending nose surface 202N of top endplate 202 with a downwardly sloped and horizontally extending nose surface 204N of bottom endplate, where the combination is segmented by an upward facing portion of seam 224.

Outer surfaces of the top and bottom endplates 202 and 204 of FIG. 2 may include osseus-friendly lattices 213 to promote osseointegration after insertion of the corresponding spinal implant assembly between vertebrae. In addition, each of top and bottom endplates of FIG. 2 define holes 215 at side surfaces thereon configured to receive fasteners, such as pins, therein, as will be explained in further detail in relation to the embodiments of FIGS. 12A-14 below.

FIGS. 3A-3C show top endplate 202 including non-planar inner surface 208 defining undulations, an outer-facing surface or top surface 218 including teeth 211. Holes 215 for fasteners in the form of pins, such as micro-machined holes, are shown at a side surface of top endplate 202. Lattices 212 are shown as being defined at the side surface of top endplate 202 as well. The undulations extend from a back region 201T to a front region 203T of the top endplate. Top endplate further defines a lumen segment 220T therethrough. At front region 203T thereof, the top endplate defines a downwardly sloped and horizontally extending surface 202N. At front region 203T, inner surface 208 of top endplate 202 includes an upwardly sloped surface 208N.

FIGS. 4A-4C show bottom endplate 204 including non-planar inner surface 210 defining undulations, an outer-facing surface or bottom surface 222 including teeth 211. Holes 215 for fasteners in the form of pins, such as micro-machined holes, are shown at a side surface of bottom endplate 204. Lattices 214 are shown as being defined at the side surface of bottom endplate 204 as well. The undulations extend from a back region 201B to a front region 203B of the bottom endplate. Bottom endplate further defines a lumen segment 220B therethrough. At front region 203B thereof, the bottom endplate defines a downwardly sloped and horizontally extending surface 204N. At front region 203B, inner surface 210 of bottom endplate 204 includes an upwardly sloped surface 210N.

The undulations of non-planar inner surface 208 conform to the undulations of non-planar inner surface 210. Grooved recess 205T of top endplate 202 and grooved recess 205B of bottom endplate 204, when the top and bottom endplates are assembled, together form grooved recess 205 of FIG. 2 to receive the front portion of a spinal implant inserter (not shown).

In the shown embodiment of FIGS. 3A-3C and FIGS. 4A-4C, sloped surface 208N has a convex shape in side profile thereof as best seen in FIG. 3A, and sloped surface 210N has a concave shape in a side profile thereof as best seen in FIG. 4A. According to some embodiments, sloped surface 208N may further have a convex configuration in a front-elevational profile thereof. Likewise, sloped surface 210N may further have a concave configuration in a front-elevational profile thereof, for example as suggested in FIG. 4A. Sloped surface 208N nests within sloped surface 210N, in this manner imparting further mechanical strength to the spinal implant assembly 200, especially during its insertion between two vertebrae.

As seen in FIG. 3A, according to some embodiments, a normal vector V_{T} to surface 208N has a horizontal component pointing forward and having a magnitude larger than a magnitude of any vertical component thereof. In the shown embodiment of FIG. 3A, surface 208N has a normal vector V_{T} which is mostly horizontal.

As seen in FIG. 4A, according to some embodiments, a normal vector V_{B} to surface 210N has a horizontal component pointing backward and having a magnitude larger than a magnitude of any vertical component thereof. In the shown embodiment of FIG. 4A, surface 210N has a normal vector V_{B} which is mostly horizontal.

Advantageously, when assembled, for example as shown in assembly 200 of FIG. 2 the upturned nose configuration as afforded by nesting surfaces 208N and 210N (see FIGS. 3A and 4A) imparts structural robustness/strength to a spinal implant assembly that incorporates the same, preventing the top and bottom endplates from separating from one another during insertion. Such strength advantageously contributes to a spinal implant assembly that includes a portion similar to nose surface 200N in order to impart further stability and mechanical strength to the spinal implant assembly during insertion/implantation.

Referring back to FIG. 1B, recall that insertion of a spinal implant assembly as described herein would involve, first, fastening the spinal implant assembly to an inserter, second, inserting the same between two vertebrae with its front/nose portion facing forward toward the spine and while the assembly is on its side, and third, twisting the inserter to turn it upright in position between the two vertebrae. Where a multi-component spinal implant assembly, such as those described herein, is to be used for such insertion, provision of an upturned nose portion such as 100N or 200N with nesting upward sloping inner surfaces of the top and bottom endplates, would provide a structural barrier to the top portion and bottom portion becoming separated from one another by virtue of shearing forces to the same during insertion, in this manner advantageously a structurally robust spinal implant assembly that is much less prone to breaking during insertion than spinal implants according to the state of the art.

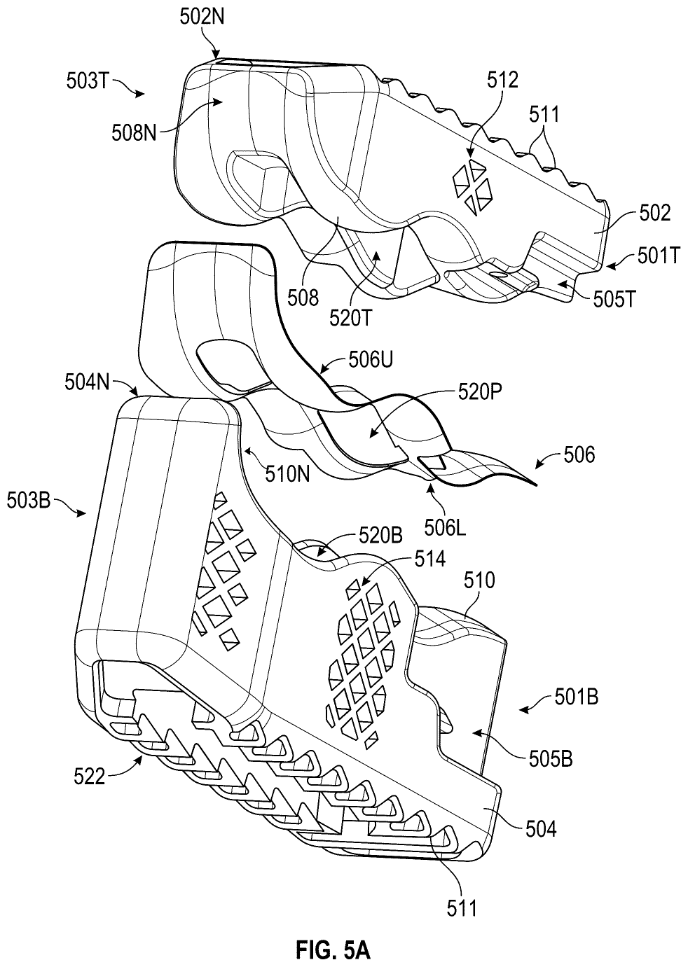

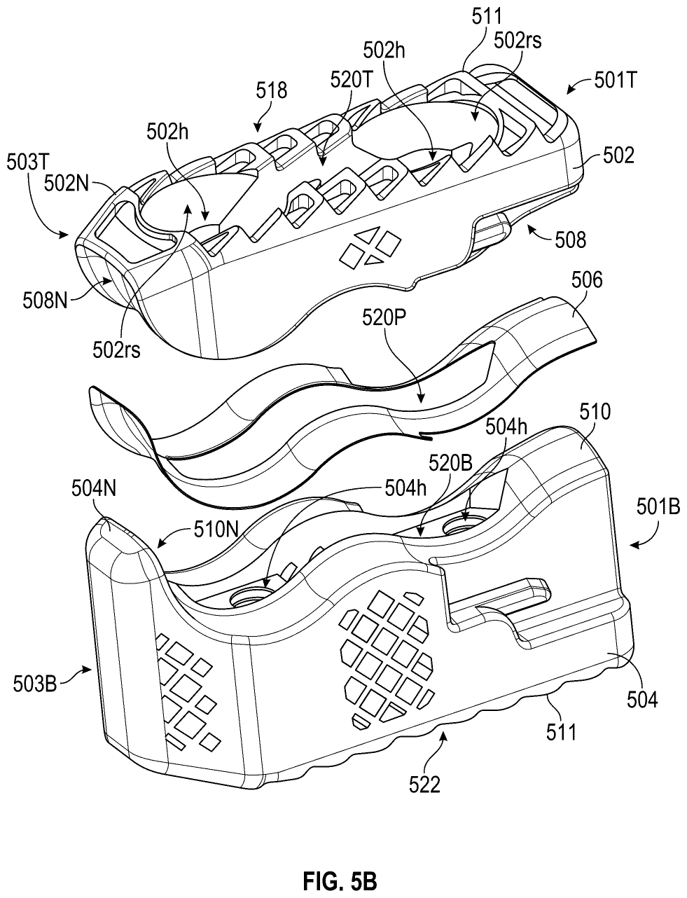

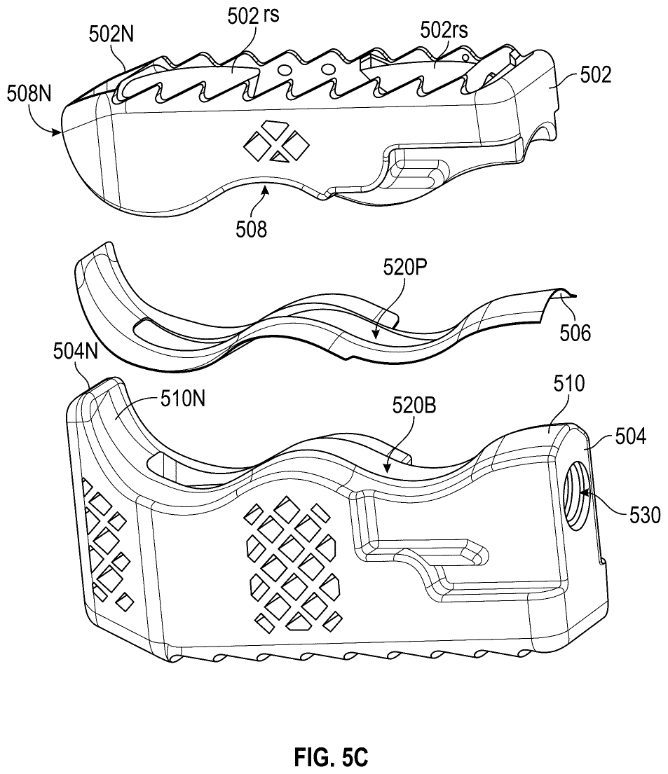

Let us now refer to FIGS. 5A-5C, which show respective exploded views of components (top and bottom endplates and a piezoelectric layer) of a spinal implant assembly prior to their assembly. In FIGS. 5A-5C, fasteners have been omitted for case of description.

The components shown in FIGS. 5A-5C may for example be used in embodiments depicted herein and described in further detail below in relation to FIGS. 8A-8C.

FIGS. 5A-5C show exploded views of top endplate 502 and bottom endplate 504 each having inner surfaces that conform to one another. FIGS. 5A-5C further show a piezoelectric layer 506 including a lumen segment therein. Upon assembly, a lumen would be defined by way of a registration of a corresponding top lumen segment 520T of the top endplate with a corresponding bottom lumen segment 520P of the piezoelectric layer and lumen segment 520B of the bottom endplate.

Piezoelectric layer 506 is shown as having a non-planar configuration with undulations and conforms, at its upper surface 506U, to the inner surface 508 of the top endplate 502, and at its lower surface 506L, to the inner surface 510 of the bottom endplate 504. However, embodiments are not so limited. Piezoelectric layer 506, in a preferred embodiment, has a planar/flat configuration prior to assembly, and is configured to assume the non-planar shape shown in FIGS. 5A-5C only upon assembly of a corresponding spinal implant assembly that incorporates the same.

Top endplate 502 includes non-planar inner surface 508 defining undulations, an outer-facing surface or top surface 518 including teeth 511. Lattices 512 are shown as being defined at the side surface of top endplate 502 as well. The undulations extend from a back region 501T to a front region 503T of the top endplate. At front region 503T thereof, the top endplate defines a downwardly sloped and horizontally extending surface 502N. At front region 503T, inner surface 508 of top endplate 502 includes an upwardly sloped surface 508N.

Bottom endplate 504 including non-planar inner surface 510 defining undulations, an outer-facing or bottom surface 522 including teeth 511. Lattices 514 are shown as being defined at the side surface of bottom endplate 504 as well. The undulations extend from a back region 501B to a front region 503B of the bottom endplate. At front region 503B thereof, the bottom endplate defines a downwardly sloped and horizontally extending surface 504N. At front region 503B, inner surface 510 of bottom endplate 504 includes an upwardly sloped surface 510N.

The undulations of non-planar inner surface 508 conform to the undulations of non-planar inner surface 510. Grooved recess 505T of top endplate 502 and grooved recess 505B of bottom endplate 504, when the top and bottom endplates are assembled, together form grooved a recess similar to grooved recess 205 of FIG. 2 to receive the front portion of a spinal implant inserter (not shown). In addition, back region 503B of the bottom endplate 504 includes a threaded hole 530 (FIG. 5C) therein to receive a threaded portion of such inserter.

According to some embodiments, such as the embodiments of FIGS. 1-8C, 12A-13B, and 15A-15E, by way of example, at a front region of the spinal implant assembly formed from components of FIGS. 5A-5C, the non-planar inner surface 508N and the non-planar inner surface 510N define a vertically biased interface (e.g., the seam or interface that is mainly vertical at the front region of spinal implant assembly 500). In this context, see also the seam 224 of FIG. 2.

Although embodiments as depicted herein in the context of FIGS. 1-8C, 12A-13B and 15A-15E depict an “upturned” nose of a spinal implant assembly where the inner surface of the bottom endplate slopes upward and the inner surface of the top endplate also slopes upward, embodiments are not so limited, and include within their scope a spinal implant assembly where the inner surface of the bottom endplate slopes downward (e.g. configured similarly to the inner surface of the shown top endplate of any of the relevant figures herein), and the inner surface of the top endplate slopes downward as well (e.g. configured similarly to the inner surface of the shown bottom endplate of any of the relevant figures herein). In any case, the interface between the top and bottom endplates at the front region of the spinal implant assembly would be vertically biased (mostly vertical, which includes fully vertical as well).

Sloped surface 508N has a convex shape in side profile thereof, and sloped surface 510N has a concave shape in a side profile thereof. According to some embodiments, sloped surface 508N may further have a convex configuration in a front-elevational profile thereof. Likewise, sloped surface 510N may further have a concave configuration in a front-elevational profile thereof.

Similar to the embodiment of FIG. 3A, a normal vector to surface 508N has a horizontal component pointing forward and having a magnitude larger than a magnitude of any vertical component thereof. In the shown embodiment, surface 508N has a normal vector which is mostly horizontal.

Similar to the embodiment of FIG. 4A, a normal vector to surface 510N has a horizontal component pointing backward and having a magnitude larger than a magnitude of any vertical component thereof. In the shown embodiment, surface 510N has a normal vector which is mostly horizontal.

Unlike the endplates in the embodiments of FIGS. 2, 3A-3C and 4A-4C, in the embodiment of FIGS. 5A-5C, the top endplate 502 defines a plurality of first holes 502h therein extending vertically, and the bottom endplate includes a plurality of second holes 504h therein extending vertically, the second holes being threaded. The top endplate 502 and the bottom endplate 504 are configured such that, in a spinal implant assembly formed therefrom, respective ones of the plurality of second holes 504h are in registration with respective corresponding ones of the plurality of first holes 502h to form respective vertical fastener holes. The vertical fastener holes to be formed by first holes and second holes are such that a screw extending vertically through a corresponding one of the vertical fastener holes has threads that engage threads of the second holes. Whether or not such a screw, in a region of the corresponding first hole, is threaded (e.g., a lag screw would not be threaded in the region of a corresponding first hole), its vertical surfaces do not engage vertical walls of the corresponding first hole in the corresponding spinal implant assembly.

In the shown embodiment of FIGS. 5A-5C, the first holes 502h in the top endplates 502, at entry regions thereof, define concave-conical-frustum-shaped ring seat surfaces 502rs, which, in the shown embodiment as best seen in FIG. 5B, are truncated in a region of lumen formed from a registration of lumen segments 520T, 520P and 520B. Ring seat surfaces 502rs are to accommodate therein lower surfaces of respective electrically insulating rings of respective fasteners, as will be explained in further detail in relation to the embodiment of FIGS. 8A-8C. Although the ring seat surfaces 502rs are truncated as shown, embodiments are not so limited, and include ring seat surfaces that are complete (extend throughout 360 degrees of the ring).

The components 502, 504 and 506 of the spinal implant assembly are configured such that, when assembled, a front portion of the spinal implant assembly includes an downwardly sloped and horizontally extending nose surface corresponding to a combination of downwardly sloped and horizontally extending nose surface 502N of top endplate 502 along with a downwardly sloped and horizontally extending nose surface 504N of bottom endplate, where the combination is segmented by an upward facing portion of a seam 624 (FIG. 6B) between top and bottom endplates.

Reference is now made to FIGS. 6A-6C, which show, respectively, a partially transparent side-elevational view, a top perspective view, and a top plan view of a spinal implant assembly 600 according to yet another embodiment. The spinal implant assembly 600 includes a top endplate 602, a bottom endplate 604 and the piezoelectric layer 606, which are configured similarly to like components of FIGS. 5A-5C, except that, in spinal implant assembly 600, the top endplate 602 has first holes 602h that, instead of convex-conical-frustum-shaped entry regions, have entry regions defined by enlarged stepped cylindrical portions 602e. Outer surfaces of the top and bottom endplates include teeth 611 thereon. A lumen 620 is defined within the spinal implant assembly 600.

A front region 603 of the spinal implant assembly 600 includes an downwardly sloped and horizontally extending nose surface 600N corresponding to a combination of downwardly sloped and horizontally extending nose surface 602N of top endplate 602 along with a downwardly sloped and horizontally extending nose surface 604N of bottom endplate, where the combination is segmented by an upward facing portion of a seam 624 between top and bottom endplates.

Grooved recesses of top and bottom endplates together form grooved recess 605 of to receive the front portion of a spinal implant inserter (not shown). In addition, back region 601 of the bottom endplate includes a threaded hole 630 therein to receive a threaded portion of such inserter.

Top endplate 602 defines a plurality of first holes 602h therein extending vertically, and the bottom endplate includes a plurality of second holes 604h therein extending vertically, the second holes being threaded. The top endplate 602 and the bottom endplate 604 are configured such that, in a spinal implant assembly formed therefrom, respective ones of the plurality of second holes 604h are in registration with respective corresponding ones of the plurality of first holes 602h to form respective vertical fastener holes 625.

The vertical fastener holes 625 formed by first holes 602h and second holes 604h are such that fasteners in the form of screws 627 extending vertically therethrough have threads that engage threads of the second holes 604h. In the shown embodiment of FIGS. 6A-6C, the screws 627 are threaded along an entirety of bodies thereof, and include an electrically insulating material, such as a polymer, such as, for example, polyether ether ketone (PEEK). Because the top and bottom endplates 602 and 604 are electrically conductive, it is important for them to be electrically isolated from one another when they are assembled in order for the piezoelectric layer therebetween to have oppositely polarized surfaces to promote osteogenesis. Thus, providing electrically insulating screws 627 allows the top and bottom endplates to be securely attached in order to prevent cracking or breakage during insertion/implantation and through the life of the implant, in order to prevent the top and bottom endplates from separating from one another, and further in order to electrically insulate the top and bottom endplates from one another to fulfill the goal of achieving desired polarization of the piezoelectric layer 606 under compressive and shear forces.

Screws used as fasteners according to embodiments are not limited to the shape as shown in FIGS. 6A-6C. A fastener in the form of a screw according to embodiments may include any screw shape, such as a lag screw, which has a threaded end and a smooth/unthreaded shaft portion below the head.

Whether or not such a screw, in a region of the corresponding first hole, is threaded (e.g., a lag screw would not include threads in the region of corresponding first hole), its vertical surfaces, according to some embodiments, are not to engage vertical walls of the corresponding first hole in the corresponding spinal implant assembly. Thus, in the shown embodiment of FIGS. 6A-6C, below enlarged stepped cylindrical portions 602e at entry regions of holes 602h, the first holes have a narrowed shaft portion 602n that is wider than a diameter of corresponding screw 627 extending therein, such that threads of screw 627 at the region of narrowed shaft portion 602n do not engage walls of the narrowed shaft portion 602n. Lower/bearing surfaces of heads 629 of screws 627 engage steps 602s defined between enlarged stepped cylindrical portions 602e and narrowed shaft portions 602n, and their threaded ends engage threaded second holes 604h, in this way exerting a lag effect between the top endplate 602 an bottom endplate 604. It is to be understood that, even where the screws 627 were lag screws with smooth upper portions and threaded lower portions, because of the narrowed shaft portions 602n of the top endplate 602, the same lag effect would take place in the spinal implant assembly using such lag screws.

According to some embodiments, in order to make a lag effect possible, where projections are provided on an outer surface of an endplate, where the outer surface accommodates a head of a screw, a distance Dₘ (see e.g., FIG. 6A) between a topmost point of the projections and a topmost point on a screw head may be larger than a thickness of the piezoelectric layer prior to its attachment into the spinal implant assembly. In this manner, screw heads would not impede a movement of the top endplate toward the bottom endplate when compressive forces from vertebrae are being applied thereto, in this manner making a differential compression (or non-uniform compressive loading) across the piezoelectric layer possible.

Advantageously, a lag effect between the top endplate and the bottom endplate of a spinal implant assembly according to embodiments allows compressive forces exerted on the top and bottom endplates by adjacent vertebrae to compress the piezoelectric layer, as the lag affect is based on tolerances between the top endplate and the bottom endplate that would allow them to move vertically with respect to one another.

According to embodiments, such lag effect may not be brought about solely by screws, but by multiple alternative embodiments of fasteners, some examples of which will be described further below in relation to FIGS. 12A-12D, 13A-13B and 15A-15D.

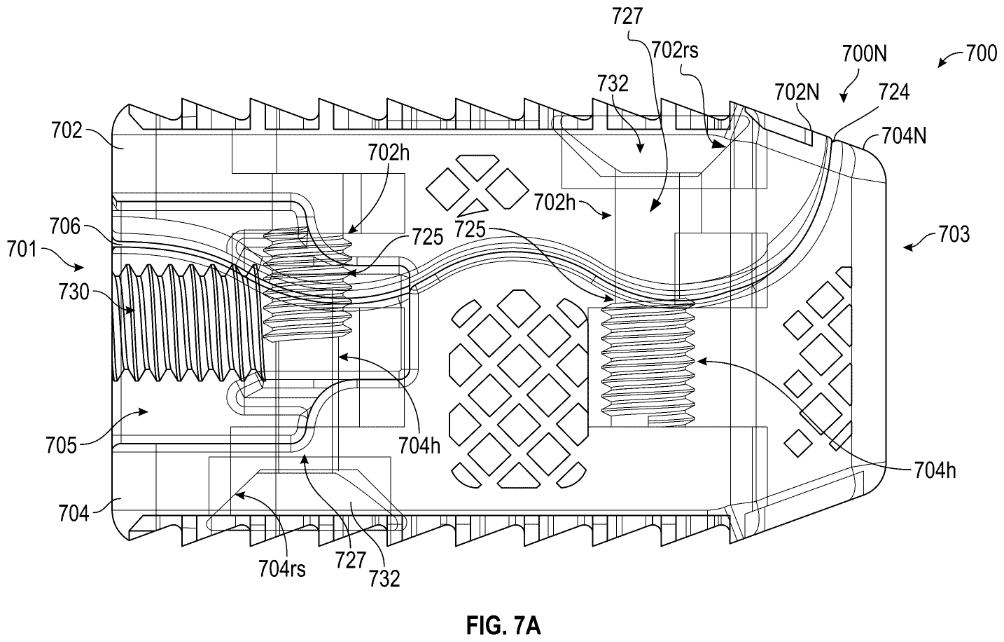

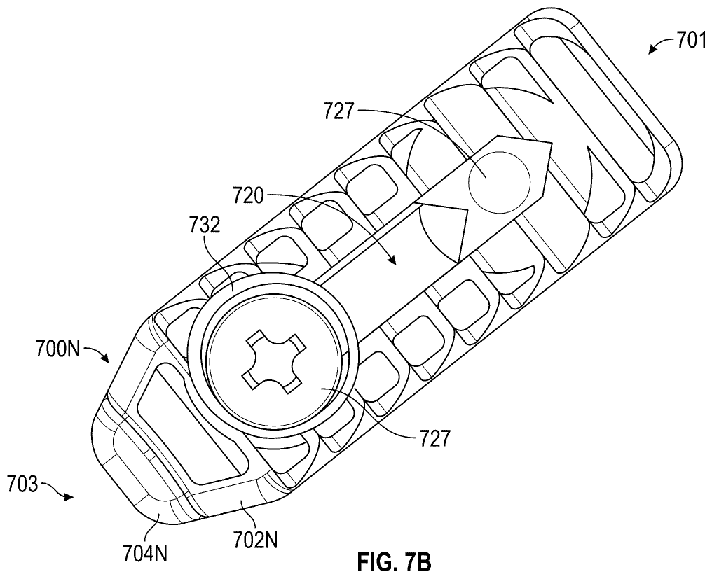

Let us now refer to the embodiment of FIGS. 7A-7B, which show, respectively, a partially transparent side-elevational view, a top perspective view, and a top plan view of a spinal implant assembly 700 according to yet another embodiment. The spinal implant assembly 700 includes a top endplate 702, a bottom endplate 704 and the piezoelectric layer 706 which are similar to the top and bottom endplates and piezoelectric layer of FIGS. 5A-5C, except that, in the embodiment of FIGS. 7A-7B, one of the two shown vertical fastener holes has an entry region at the bottom endplate and a threaded region at the top endplate. A lumen 720 defines a hollow region inside the spinal implant assembly 700 (FIG. 7B).

A front region 703 of the spinal implant assembly 700 includes an downwardly sloped and horizontally extending nose surface 700N corresponding to a combination of downwardly sloped and horizontally extending nose surface 702N of top endplate 702 along with a downwardly sloped and horizontally extending nose surface 704N of bottom endplate, where the combination is segmented by an upward facing portion of a seam 724 between top and bottom endplates.

Grooved recesses of top and bottom endplates together form grooved recess 705 of to receive the front portion of a spinal implant inserter (not shown). In addition, back region 703 of the bottom endplate includes a threaded hole 730 therein to receive a threaded portion of such inserter.

The top endplate 702 and the bottom endplate 704 are configured such that, in the spinal implant assembly formed therefrom, respective ones of the plurality of second holes 704h in the bottom endplate 704 are in registration with respective corresponding ones of the plurality of first holes 702h in the top endplate 702 to form respective vertical fastener holes 725.

Top endplate 702 defines two first holes 702h therein extending vertically, and the bottom endplate includes two second holes 704h therein extending vertically. In the shown embodiment, the second hole (in the bottom endplate 704) at a front region 703 (front second hole) of the spinal implant assembly is threaded, and the first hole (in the top endplate) at the front region 703 (front first hole) is not threaded and has smooth walls. In the shown embodiment, the second hole (in the bottom endplate 704) at a back region 701 (back second hole) of the spinal implant assembly is not threaded and has smooth walls, and the first hole (in the top endplate) at the back region 701 (front first hole) is threaded.

Fasteners in the embodiment of FIGS. 7A-7B include a pair of screws 727, and a pair of complementary rings 732, the screws 727 including a metal, such as, for example, titanium or tungsten, and the rings including an electrically insulating material, such as, for example, PEEK. In the shown embodiment of FIGS. 7A-7B, the screws 727 are electrically conductive.

The vertical fastener holes 725 formed by first holes 702h and second holes 704h are such that fasteners in the form of screws 727 extending vertically therethrough have threads that engage threads of the front second hole 704h and threads of the back first hole 702h, respectively. In the shown embodiment of FIGS. 7A-7B, the screws 727 correspond to lag screws with threaded ends and smooth shafts between the threaded ends and heads thereof.

Because the top and bottom endplates 702 and 704 are electrically conductive, it is important for them to be electrically isolated from one another when they are assembled in order for the piezoelectric layer therebetween to have oppositely polarized surfaces in order to promote osteogenesis.

In the shown embodiment of FIGS. 7A-7B, the front first holes and the back second holes, at entry regions thereof, define concave-conical-frustum-shaped ring seat surfaces 702rs and 704rs, respectively. Ring seat surfaces 702rs and 704rs, similar to the ring seat surfaces 502rs of FIGS. 5A-5C, accommodate therein, in a conforming manner, lower surfaces of the respective electrically insulating rings 732.

Advantageously, providing electrically insulating rings between respective screw heads of electrically conductive screws and a corresponding one of the top endplate and the bottom endplate the allows the top and bottom endplates to be securely attached in a manner to impart mechanical strength and reliability to the implant assembly during and after implantation, in order to prevent the top and bottom endplates from separating from one another during insertion, and further in order to electrically insulate the top and bottom endplates from one another to fulfill the goal of achieving desired polarization of the piezoelectric layer 706 under compressive and shear forces. The use of screws including metal advantageously provides further mechanical robustness to a spinal implant assembly as opposed to using screws that include an electrically insulating material, such as PEEK.

Let us now refer to the embodiment of FIGS. 8A-8C, which show, respectively, a partially transparent side-elevational partially exploded view, a top perspective partially exploded view, and a perspective front cross-sectional view a spinal implant assembly 800 according to yet another embodiment. The spinal implant assembly 800 includes a top endplate 802, a bottom endplate 504 and the piezoelectric layer 506 which correspond to the top and bottom endplates and piezoelectric layer of FIGS. 5A-5C, except that, in FIGS. 8A-8C, ring seat surfaces are not truncated. For the latter reason, parts of the top and bottom endplates and of the piezoelectric layer of FIGS. 8A-8C are marked with the very same reference numerals as those of FIGS. 5A-5C except for the ring seat surfaces 802rs, and will not necessarily be expressly mentioned in the context of the description of FIGS. 8A-8C.

Fasteners in the embodiment of FIGS. 8A-8C include a pair of screws 827, and a pair of rings 832, the screws 827 including a metal, such as, for example, titanium or tungsten, and the rings including an electrically insulating material, such as, for example, PEEK. In the shown embodiment of FIGS. 8A-8C, the screws 827 are electrically conductive and the rings 832 are electrically insulating, similar to the screws and rings of the embodiment of FIGS. 7A-7B. However, in the embodiment of FIGS. 8A-8C, the screws 827 are threaded along an entirety of bodies thereof.

Lower/bearing surfaces of heads 829 of screws 827 define convex-conical-frustum-shaped surfaces that conform to and are adjacent to the concave-conical-frustum-shaped upper surfaces of ring 832, and their threaded ends engage threaded second holes 804h, in this way exerting a lag effect between the top endplate 802 an bottom endplate 504. It is to be understood that, even where the screws 827 were lag screws with smooth upper portions and threaded lower portions, because of the narrowed shaft portions 802n of the top endplate 802, the same lag effect would take place in the spinal implant assembly using such lag screws.

Similar to prior embodiments as described herein, vertical surfaces of screws 827 are not to engage vertical walls of the corresponding first holes 802h.

Thus, as best seen in the alternative embodiment of FIG. 8C (where a lag screw 827′ is shown instead of the fully threaded screw 827 of FIGS. 8A and 8B), the first holes 502h have a narrowed shaft portion 802n that is wider than a diameter of corresponding screw 827′ extending therein, such that threads of the shown screw at the region of the narrowed shaft portion do not engage walls of the narrowed shaft portion.

Rings according to embodiments, and as best seen in FIG. 8C, are configured to form a physical barrier between a fastener, such as a screw, and the endplate accommodating a head of such fastener, such as the head of said screw.

In the shown embodiment, the first holes 502h in the top endplate 502, at entry regions thereof, define concave-conical-frustum-shaped ring seat surfaces 802rs. Ring seat surfaces 802rs are similar to ring seat surfaces 502rs of FIGS. 5A-5C, except that they are not truncated. Ring seat surfaces 802rs are adjacent to and conform to convex-conical-frustum-shaped lower surfaces 8321 of respective electrically insulating rings 832 of respective fasteners. As best seen in FIG. 8C, the ring 832 serves as a barrier between the screw 827′ and the top endplate 802.

Referring now to FIG. 9, a top perspective view is provided of an embodiment of a fastener including a lag screw 927 and a ring 932. Lag screw 927 may be similar to lag screw 827′ of FIG. 8C, and ring 932 may have a same configuration as rings 732 and/or 832 as described above. Lag screw 927 has threaded end 927e, a head 927h, and a smooth/unthreaded shaft portion 927s below the head 927h. A lower surface 9271 (or bearing surface) at an underside of head 927h is convex-conical-frustum-shaped, and is to conform to and be adjacent a corresponding concave-conical-frustum-shaped ring seat surface at an entry region of an opening in an endplate of a spinal implant assembly, as depicted for examples in FIGS. 7A-7B or in FIGS. 8A-8C.

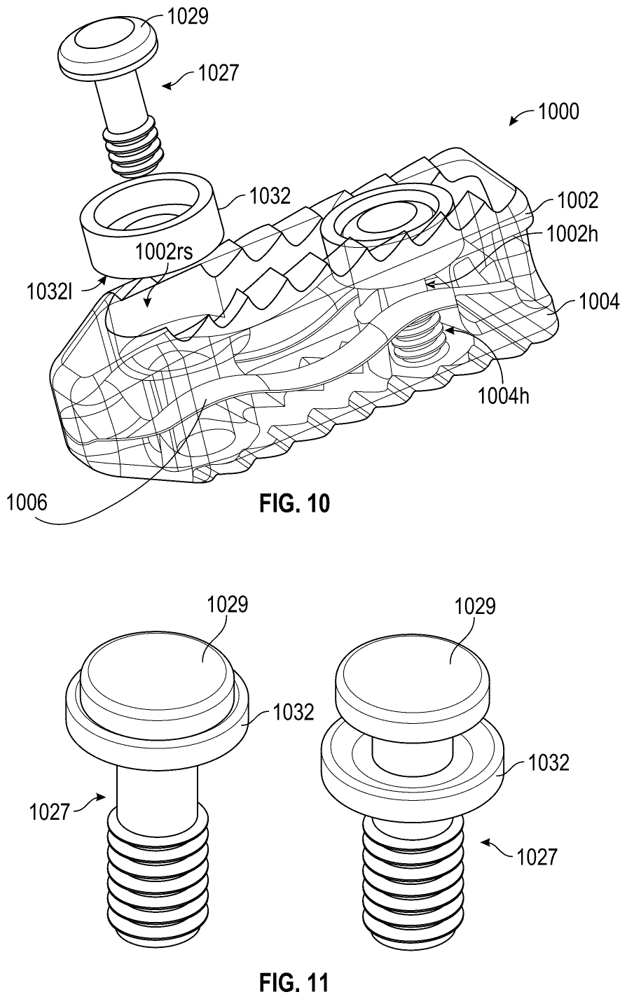

Let us now refer to FIGS. 10 and 11, which show, respectively, a partially transparent side perspective partially exploded view of a spinal implant assembly 1000 according to yet another embodiments, and a side perspective view of a pair of fasteners to be used in the spinal implant assembly 1000 of FIG. 10.

The spinal implant assembly 1000 includes a top endplate 1002, a bottom endplate 1004 and the piezoelectric layer 1006 which correspond to the top and bottom endplates and piezoelectric layer of FIGS. 5A-5C, except for the shape of the bearing seat surfaces and associated rings, and except for the configuration of the nose portion of the assembly.

Fasteners in the embodiment of FIGS. 10 and 11 include a pair of lag screws 1027, and a pair of rings 1032, the screws 1027 including a metal, such as, for example, titanium or tungsten, and the rings including an electrically insulating material, such as, for example, PEEK. In the shown embodiment of FIGS. 10-11, the screws 1027 are electrically conductive and the rings 1032 are electrically insulating, similar to the screws and rings of the embodiment of FIGS. 7A-7B and 8A-8C. However, in the embodiment of FIGS. 10-11, the screws have flat bearing surface under heads thereof

Lower/bearing surfaces of heads 1029 of screws 1027 define flat annulus-shaped surfaces that conform to and are adjacent to upper surfaces of ring 1032, and their threaded ends engage threaded second holes 1004h, in this way exerting a lag effect between the top endplate 1002 and bottom endplate 1004.

Rings according to embodiments, and as best seen in FIG. 11, are configured as concave collared annulus-shaped rings to form a physical barrier between a fastener, such as a screw 1027, and the endplate accommodating a head of such fastener, such as the head of said screw.

In the shown embodiment, the first holes 1002h in the top endplates 1002, at entry regions thereof, define concave collared annulus-shaped ring seat surfaces 1002rs. Ring seat surfaces 1002rs are adjacent to and conform to convex annulus-shaped lower surfaces 10321 of respective electrically insulating rings 1032 of respective fasteners. A ring 1032 serves as a barrier between the corresponding screw 1027 and the top endplate 1002.

Let us now refer to the embodiment of FIGS. 12A-12D, which show, respectively, a perspective side-elevational view of a spinal implant assembly, and three respective stages of assembly of the spinal implant assembly in perspective view. The spinal implant assembly 1200 includes a top endplate 202, a bottom endplate 204 and the piezoelectric layer 206 which correspond to the top and bottom endplates and piezoelectric layer of FIGS. 2, 3A-3B and 4A-4B. For the latter reason, parts of the top and bottom endplates and of the piezoelectric layer of FIGS. 12A-12D are marked with the very same reference numerals as those of FIG. 2, and will not necessarily be expressly mentioned in the context of the description of FIGS. 12A-12D.

Fasteners in the embodiment of FIGS. 12A-12D include a plurality of connecting brackets 1233 extending vertically within the lumen 220 (FIG. 12B), which, as noted previously, corresponds to a registration of a lumen segment 220T of top endplate and a lumen segment 220B of bottom endplate. The connecting brackets 1233 of FIGS. 12A-12D include an electrically insulating material, such as, for example, PEEK. Individual ones of the plurality of connecting brackets have flat side surfaces and rounded top and bottom ends.

Each of the plurality of connecting brackets defines a top hole 1233Th (FIG. 12D) therein and a bottom hole 1233Bh therein, the top hole 1233Th in registration with corresponding ones of the plurality of holes 215 of the top endplate 202 (hereinafter first holes 215T), and the bottom hole 1233Bh in registration with corresponding ones of the plurality of holes 215 of the bottom endplate 204 (hereinafter second holes 215B). The fasteners further include a plurality of top pins 1237T and a plurality of bottom pins 1237B. Respective ones of the plurality of top pins 1237T extend through the top hole 1233Th of a corresponding one of said each of the plurality of connecting brackets 1233 and through corresponding ones of the plurality of first holes 215T. Respective ones of the plurality of bottom pins 1237B extend through the bottom hole 215B of a corresponding one of said each of the plurality of connecting brackets 1233 and through corresponding ones of the plurality of second holes 215B.

Pins 1237T and 1237B may include a metal or an electrically insulating material, or both. Such pins may be either electrically conductive, or electrically insulating. A choice of material of pins in the embodiment of FIGS. 12A-12D is not limited by whether or not such material is electrically conductive, by virtue of the fact that brackets 1233, themselves electrically insulating, ensure an electrical isolation of the top endplate from the bottom endplate.

Because the top and bottom endplates 202 and 204 are electrically conductive, it is important for them to be electrically isolated from one another when they are assembled in order for the piezoelectric layer therebetween to have oppositely polarized surfaces to promote osteogenesis. Thus, providing electrically insulating connecting brackets 1233 allows the top and bottom endplates to be securely attached in order to, in order to prevent the top and bottom endplates from separating from one another, and further in order to electrically insulate the top and bottom endplates from one another to fulfill the goal of achieving desired polarization of the piezoelectric layer 206 under compressive and shear forces.

For the assembly of components of spinal implant assembly 1200, as depicted in FIG. 12B, the top endplate 202, piezoelectric layer 206 and bottom endplate 204 are first aligned as shown. Next, as depicted in FIG. 12C, the PEEK brackets 1233 are aligned within the lumen 220 such that holes thereof almost register with corresponding holes of the top endplate and of the bottom endplate. Thereafter, as shown in FIG. 12D, a compressive force is applied pressing the top endplate and the bottom endplate toward one another. FIG. 12D thus suggests a “preloading” of the spinal implant assembly before effecting an attachment of components thereof. By way of example, such compressive forces may be between about 20 pounds to about 30 pounds. The preloading may register respective ones of the top holes 1233Th with corresponding ones of the first holes 215T, and may register respective ones of the bottom holes 215B with corresponding ones of the second holes 215B. As compressive forces are being applied, as shown in FIG. 12D, pins 1237T and 1237B may be inserted into corresponding holes of the brackets and of the top and bottom endplates to secure the components of the spinal assembly together.

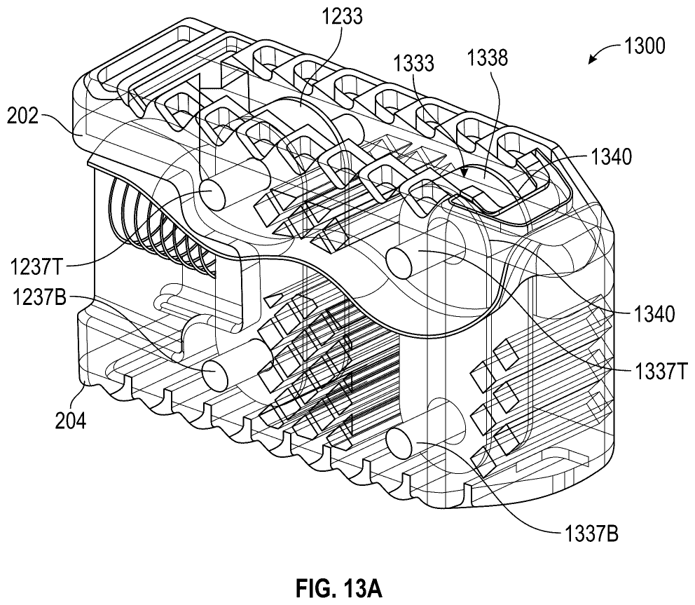

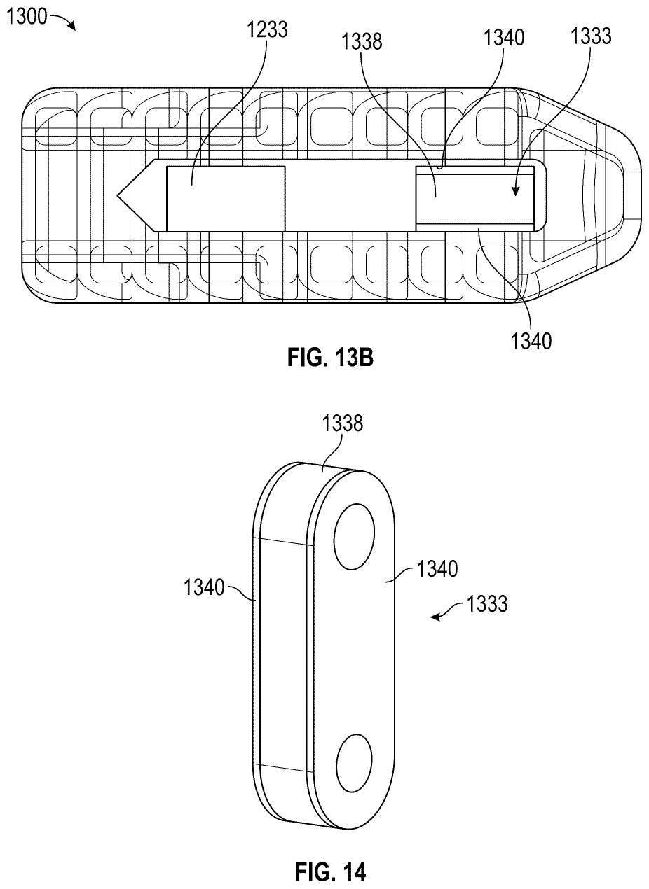

Let us now refer to the embodiment of FIGS. 13A-13B and 14, which pertain to yet another embodiment in the form of spinal implant assembly 1300. Spinal implant assembly 1300 is identical to spinal implant assembly 1200 of FIGS. 12A-12D, except for the fact that one of its brackets 1333 includes a metal core 1338 that is covered at flat sides thereof with electrically insulating cover layers 1340. The cover layers 1340 may for example include PEEK, and, as best seen in FIG. 13B, provide an electrically insulating barrier between metal core 1338 and adjacent parts of the top and bottom endplates, in this manner ensuring an electrical isolation of top and bottom endplates with respect to one another.

Pins 1337T and 1337B for bracket 1333 include an electrically insulating material, such as PEEK. A choice of material of pins 1337T and 1337B in the embodiment of FIGS. 13A-13B and 14 is limited to electrically insulating materials, by virtue of the fact that brackets 1333, are themselves electrically conductive, and the use of cover layer 1340 and of electrically insulating pins ensure an electrical isolation of the top endplate from the bottom endplate.

Although the embodiment of FIGS. 13A-13B and 14 shows a single one of the pair of shown brackets as including metal, embodiments include within their scope the use of all brackets of a spinal implant assembly that may include a metal core comprising metal, and cover layers on the metal core.

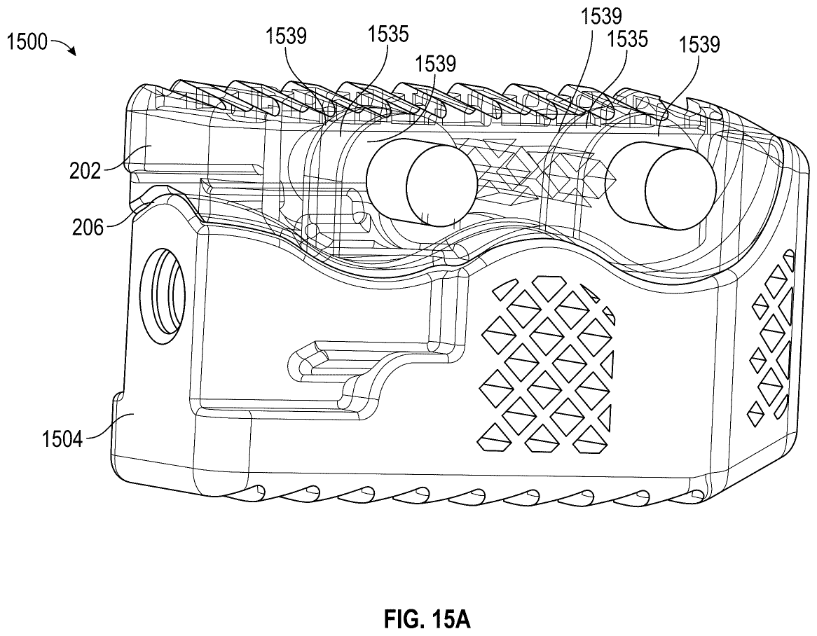

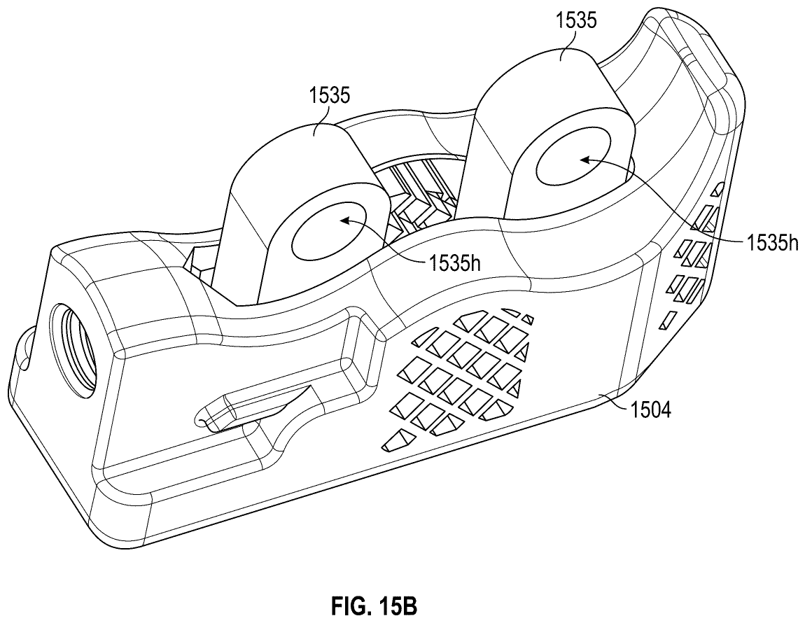

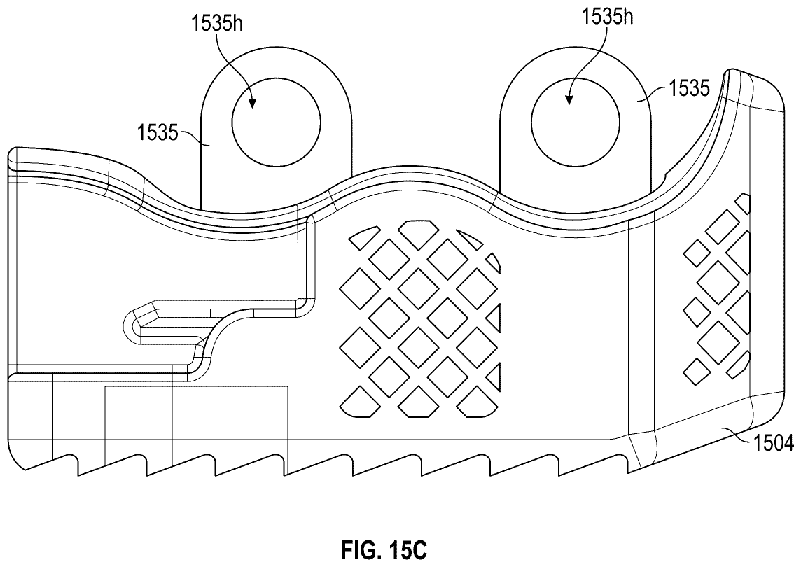

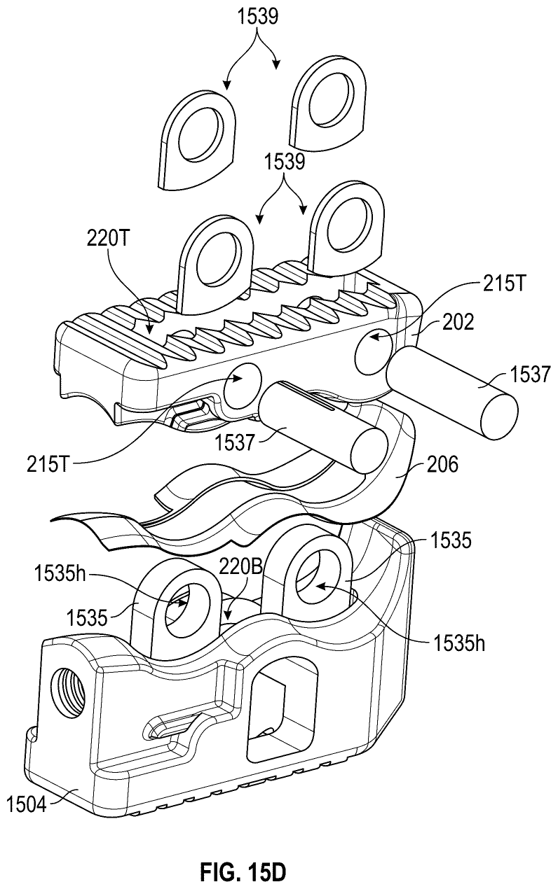

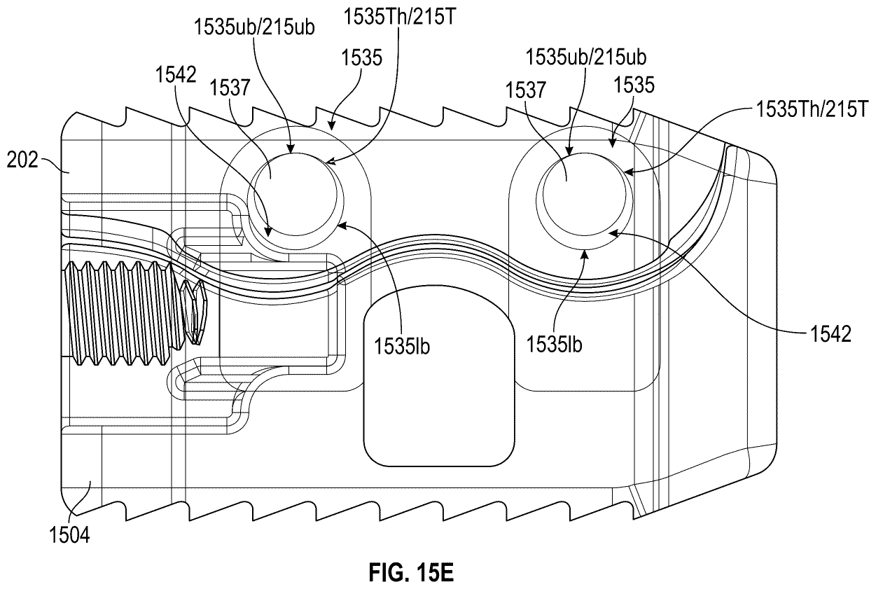

Let us now refer to the embodiment of FIGS. 15A-15D, which pertain to yet another embodiment in the form of spinal implant assembly 1500. Spinal implant assembly 1500 is identical to spinal implant assembly 1200 of FIGS. 12A-12D, except for the fact that, instead of brackets, spinal implant assembly 1500 provides as fasteners among other things including a pair of struts 1535 extending vertically upward from an inner surface of the bottom endplate, and a pair of electrically insulating washers 1539 flanking lateral surfaces of the struts to electrically insulate the same from the top endplate 202. The spinal implant assembly 1500 includes a top endplate 202, a bottom endplate 1504 and the piezoelectric layer 206 which correspond to the top and bottom endplates and piezoelectric layer of FIGS. 2, 3A-3B and 4A-4B. For the latter reason, parts of the top and bottom endplates and of the piezoelectric layer of FIGS. 15A-15D are marked with the very same reference numerals as those of FIG. 2, and will not necessarily be expressly mentioned in the context of the description of FIGS. 15A-15D.

Fasteners in the embodiment of FIGS. 15A-15D include a plurality of connecting struts 1535 extending vertically within the lumen 220 and having at their base the inner surface 210 of bottom endplate 204. The lumen, as noted previously, corresponds to a registration of a lumen segment 220T of top endplate and a lumen segment 220B of bottom endplate. The connecting struts 1535 of FIGS. 15A-15D may be unitary (one-piece) with the bottom endplate 204, and may thus include an electrically conductive material, such as titanium or tungsten by way of example. The struts may each have flat side surfaces and rounded top ends.

Each of the plurality of connecting struts defines a hole 1535h therein, the hole 1535h in registration with corresponding ones of the plurality of holes 215 of the top endplate 202 (hereinafter first holes 215T). The fasteners further include a plurality of pins 1537. Respective ones of the plurality of pins 1537 extend through the hole 1535h of a corresponding one of said each of the plurality of connecting struts 1535 and through corresponding ones of the plurality of first holes 215T. Pins 1537 may include an electrically insulating material, such as PEEK. Washers 1539 are provided at each lateral side of the struts to electrically insulate the same from the top endplate.

Because the top and bottom endplates 202 and 204 are electrically conductive, it is important for them to be electrically isolated from one another when they are assembled in order for the piezoelectric layer therebetween to have oppositely polarized surfaces to promote osteogenesis. Thus, providing connecting struts 1535 and associated washers 1539 and pins 1537 allows the top and bottom endplates to be securely attached in order to prevent cracking or breakage during insertion/implantation and through the life of the implant, in order to prevent the top and bottom endplates from separating from one another, and further in order to electrically insulate the top and bottom endplates from one another to fulfill the goal of achieving desired polarization of the piezoelectric layer 206 under compressive and shear forces.

For the assembly of components of spinal implant assembly 1500, as depicted in FIG. 15D, the top endplate 202, piezoelectric layer 206 and bottom endplate 1504 are first aligned as shown. Next, the struts 1535 and washers 1539 are aligned within the lumen 220 such that holes thereof almost register with corresponding holes of the top endplate. Thereafter, a compressive force is applied pressing the top endplate and the bottom endplate toward one another. A “preloading” of the spinal implant assembly is therefore implemented before effecting an attachment of components thereof. By way of example, such compressive forces may be between about 20 pounds to about 30 pounds. The preloading may register respective ones of the top holes 1535Th with corresponding ones of the first holes 215T. As compressive forces are being applied, pins 1537 may be inserted into corresponding holes of the struts and of the top endplate to secure the components of the spinal assembly together.

Reference is now made in particular to FIG. 15E, which show respective ones of the plurality of pins 1537 abutting an upper boundary 1535ub of the hole 1535h of a corresponding one of struts 1535 and an upper boundary 215ub of a corresponding one of the first holes 215T, and further defining a gap 1542 with respect to a lower boundary 15351b of said hole 1535h and with respect to a lower boundary of the corresponding one of the first holes. Gaps 1542 make possible a lag effect between the top endplate 202 and bottom endplate 204 by allowing them to move vertically with respect to one another based on tolerances afforded by the gap 1542 when compressive (or axial) forces are exerted on the top and bottom endplates.

According to some embodiments, similar gaps may exist for the brackets 12A to 14, where: each top pins abuts an upper boundary of a top hole of a corresponding bracket and an upper boundary of a corresponding first hole; each top pin further defines a gap with respect to a lower boundary of said top hole and with respect to a lower boundary of the corresponding first hole; each bottom pins abuts a lower boundary of a bottom hole of a corresponding bracket and an lower boundary of a corresponding second hole; and each bottom pin further defines a gap with respect to an upper boundary of said bottom hole and with respect to a upper boundary of the corresponding second hole. Such gaps make possible a lag effect between the top endplate and the bottom endplate by allowing them to move vertically with respect to one another based on tolerances afforded by the gap when compressive (or axial) forces are exerted on the top and bottom endplates.

Advantageously, as noted previously, a lag effect between the top endplate and the bottom endplate of a spinal implant assembly according to embodiments allows compressive forces exerted on the top and bottom endplates by adjacent vertebrae to compress the piezoelectric layer, as the lag affect is based on tolerances between the top endplate and the bottom endplate that would allow them to move vertically with respect to one another.

Although the embodiment of FIGS. 15A-15D shows struts emanating from the inner surface of the bottom endplate, embodiments include within their scope the provision of struts that emanate from the inner surface of the top endplate instead, or of one or more struts that emanate from inner surfaces of respective ones of the top endplate and the bottom endplate.

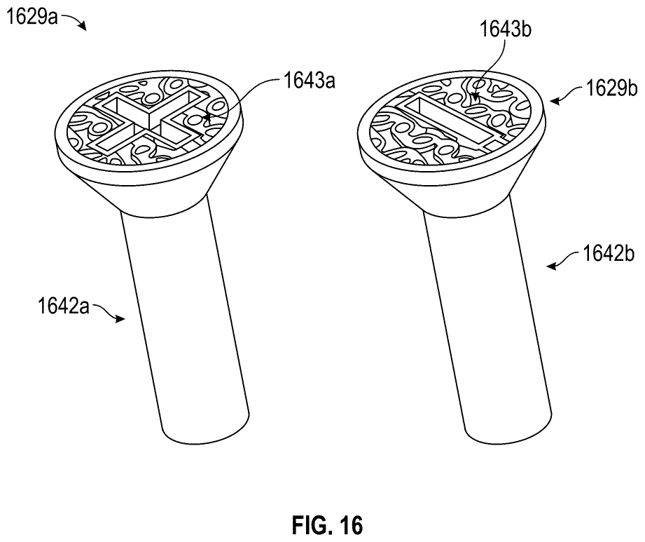

Referring now to FIG. 16, alternative structures 1642a and 1642b are shown for screw heads of screws to be used as fasteners in a spinal implant assembly according to some embodiments, such as for screws of any of the embodiments of FIGS. 6A-11 as described above. Screw structures 1642a and 1642b show head portions of screws according to some embodiments, although, in the shown structures, the threaded portions of such screws are not shown for case of description. Although the screw heads in FIG. 16 are shown as including a convex-conical-frustum-shaped bearing surface under heads thereof, embodiments are not so limited, and include within their scope screw bearing surfaces that have any shape.

As seen by way of structures 1642a and 1642b, the heads 1629a and 1629b of screws according to some embodiments may include osseus friendly lattices 1643a and 1643b on upper surfaces thereon. The osseus friendly lattices advantageously serve to further osteogenesis after implantation of the associated spinal implant assembly between two vertebrae. According to an alternative, a screw according to some embodiments may be a hollow screw with a head featuring an osseous-friendly surface designed with perforations or apertures that are in communication with the hollow shaft of the screw. This design allows for bone growth through the holes of the osseus friendly lattice of the screw, promoting osteointegration and facilitating the interaction between the surrounding bone tissue and the implant assembly.

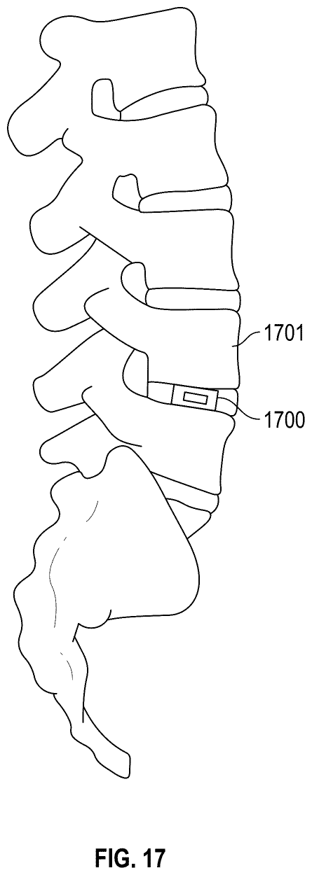

Referring now to FIG. 17, a spinal column 1701 of a human is shown including a spinal implant assembly 1700 inserted therein, the spinal implant assembly corresponding to any of the spinal implant assemblies according to the embodiments described herein. Although a human spine is depicted in FIG. 17, a spinal implant assembly according to embodiments may be inserted into the spine of any animal.

In some of the shown embodiments, although fasteners (such as screws, brackets or struts) are depicted as included a pair of fasteners that are spaced from one another in a longitudinal direction (between the front region and the back region of a spinal implant assembly), embodiments are not so limited, and include within their scope the provision of one, two or more fasteners in a longitudinal direction and/or in a transverse direction (a horizontal direction perpendicular to the longitudinal direction) of a spinal implant assembly.

In some of the shown embodiments, although holes within top and/or bottom endplates or within brackets are sometimes depicted as having a cylindrical cross section, embodiments are not so limited, and include within their scope the provision of holes of any cross section, such as oval, polygonal, rectangular, square, star-shaped, etc.

In some of the shown embodiments, although pins are depicted as having a cylindrical cross section, embodiments are not so limited, and include within their scope the provision of pins of any cross section, such as oval, polygonal, rectangular, square, star-shaped, etc.

Although embodiments as described herein show a spinal implant assembly with an upturned nose portion or upturned nose configuration, for example as depicted in relation to most figures, except for example in FIG. 10, embodiments are not so limited, and include within their scope a spinal implant assembly without an upturned nose portion, but including any of the fasteners solutions as shown and described herein.

Although embodiments as described herein show a dual endplate spinal implant assembly, embodiments are not so limited, and include within their scope any of the endplate configurations and/or fastener solutions as shown as described herein wherein the spinal implant assembly includes top and bottom endplates, plus intermediate bodies distinct from a piezoelectric layer in between the top and bottom endplates.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out embodiments described herein.

All percentages, parts, proportions, and ratios as used herein, are by weight of the total composition, unless otherwise specified. All such weights as they pertain to listed ingredients are based on the active level and, therefore; do not include solvents or by-products that may be included in commercially available materials, unless otherwise specified.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

As used herein, “electrically conductive” in some examples may refer to a property of a material having an electrical conductivity greater than or equal to 10⁶ Siemens per meter (S/m) at 20 degrees Celsius. Examples of such materials include titanium (with relatively low electrical conductivity compared to many other metals, that is, about 2.4×10⁶ S/m) and tungsten (with much higher electrical conductivity of about 1.8×10⁷ S/m).

As used herein, an “electrically insulating material” may include a polymer, such as, for example, PEEK (Polyether Ether Ketone), Polyimide (PI), Polyphenylene Sulfide (PPS), Polyaryletherketone (PAEK), Polyetherketone (PEK), Polyetherketoneketone (PEKK), and/or Fluoropolymers.

As used herein, holes that “register” or that are in “registration” with one another are holes that are either in full registration (holes that overlap one another fully) with one another, or in partial registration with one another (holes that overlap one another partially).

Holes as described herein may be through holes or blind holes. Such holes may be provided, for example, by way of machining, or using any other suitable technique.

Top and bottom endplates, fasteners such as screws, pins and/or brackets as described herein may be unitary (i.e., a one piece component) or they may have multiple attached components that together form such components. Such components may be formed, for example, using 3D printing, or any other suitable technique.

Any one of the piezoelectric layers as described herein may be substantially planar in shape prior to assembly, and deformed into a non-planar shape, such as having undulations, waves, zig-zags, curves, creases, and/or bends, due to the application of a force to the top and bottom endplates. The piezoelectric components may conform to the inner surfaces of the endplates. The force may be the force applied due to assembly, a compression force, (such as a spinal compression force) or a shear force, or a combination of such forces. The force may be about 10 N, 25 N, 50 N, 100 N, 200 N, 500 N, 700 N, 1000 N, 1500 N, 3000 N, 5000 N, or 10,000 N. The force may be variable and range between an upper and lower bound. The electrical output may include a voltage of about 0.1 mV, 0.3 mV, 0.5 mV, 1 mV, 10 mV, 25 mV, 100 mV, 200 mV, 500 mV, 700 mV, 1 V, 2 V, 3 V, 4 V, 5 V, 6 V, 7 V, 10 V, 15 V, or 20 V. The electrical voltage output may be variable and range between an upper and lower bound. The electrical output may include a current of about 50 nA, 100 nA, 500 nA, 1000 nA, 3000 nA, 5000 nA, 7000 nA, 10,000 nA, 50,000 nA, 100,000 nA, 250,000 nA, 500,000 nA, 750,000 nA, 1 mA, 5 mA, 10 mA, 15 mA, 25 mA, 35 mA, or 50 mA. The electrical current output may be variable and range between an upper and lower bound. All ranges between any of the above values are hereby disclosed.

Throughout the specification, and in the claims, the terms “coupled” or “connected” mean a direct or indirect connection, such as a direct electrical, mechanical, or magnetic connection between the elements that are connected or an indirect connection, through one or more passive or active intermediary devices. The term “signal” may refer to at least one current signal, voltage signal, magnetic signal, or data/clock signal. The meaning of “a,” “an,” and “the” include plural references. The meaning of “in” includes “in” and “on.”

The terms “substantially,” “close,” “approximately,” “near,” and “about,” generally refer to being within +/−10% of a target value (unless specifically specified). Unless otherwise specified the use of the ordinal adjectives “first,” “second,” and “third,” etc., to describe a common object, merely indicate that different instances of like objects are being referred to, and are not intended to imply that the objects so described must be in a given sequence, either temporally, spatially, in ranking or in any other manner, and are not intended to imply that the objects so described must necessarily be made of different materials or have different dimensions.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of embodiments has been described with reference to specific example embodiments, various modifications and changes may be made to these embodiments without departing from the broader scope of embodiments of the present disclosure. Such embodiments of the inventive subject matter may be referred to herein, individually or collectively, by the term “invention” merely for convenience and without intending to voluntarily limit the scope of this application to any single disclosure or inventive concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

It will also be understood that, although the terms “first,” “second,” and so forth may be used herein to describe various elements, these elements should not be limited by these terms. These terms are only used to distinguish one element from another. For example, a first contact could be termed a second contact, and, similarly, a second contact could be termed a first contact, without departing from the scope of the present example embodiments. The first contact and the second contact are both contacts, but they are not the same contact.

As used herein the terms “top,” “bottom,” “upper,” “lower,” “lowermost,” and “uppermost” when used in relationship to one or more elements are intended to convey a relative rather than absolute physical configuration. Thus, an element described as an “uppermost element” or a “top element” in a device may instead form the “lowermost element” or “bottom element” in the device when the device is inverted. Similarly, an element described as the “lowermost element” or “bottom element” in the device may instead form the “uppermost element” or “top element” in the device when the device is inverted.

The description may use perspective-based descriptions such as top/bottom, in/out, over/under, and the like. Such descriptions are merely used to facilitate the discussion and are not intended to restrict the application of embodiments described herein to any particular orientation.

As used in the description of the example embodiments and the appended examples, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

In embodiments, the phrase “A is located on B” means that at least a part of A is in direct physical contact or indirect physical contact (having one or more other features between A and B) with at least a part of B.

In the instant description, “A is adjacent to B” means that at least part of A is in direct physical contact with at least a part of B.

In the instant description, “B is between A and C” means that at least part of B is in or along a space separating A and C and that the at least part of B is in direct or indirect physical contact with A and C.

In the instant description, “A is attached to B” means that at least part of A is mechanically attached to at least part of B, either directly or indirectly (having one or more other features between A and B).

In the instant description, “the As are coupled to the Bs” means that at least some of the As are coupled to at least some of the Bs, and not necessarily that all As are coupled to at least one B and all Bs are coupled to at least one A.

In the instant description, “A is within B” means that at least some of A is encompassed within the physical boundaries of B.

The use of reference numerals separated by a “/”, such as “102/104” for example, is intended to refer to 102 or 104 as appropriate. Otherwise, the forward slash (“/”) as used herein means “and/or.”

When used to describe a range of dimensions, the phrase “between X and Y” represents a range that includes X and Y. Although certain elements may be referred to in the singular herein, such elements may include multiple sub-elements. For example, “an insulating material” may include one or more insulating materials. As used herein, a “conductive contact” may refer to a portion of conductive material (e.g., metal) serving as an electrical interface between different components; conductive contacts may be recessed in, flush with, or extending away from a surface of a component, and may take any suitable form (e.g., a conductive pad or socket, or portion of a conductive line or via).

The use of the techniques and structures provided herein can be detected using tools such as: electron microscopy including scanning/transmission electron microscopy (SEM/TEM), scanning transmission electron microscopy (STEM), nano-beam electron diffraction (NBD or NBED), and reflection electron microscopy (REM); composition mapping; x-ray crystallography or diffraction (XRD); energy-dispersive x-ray spectroscopy (EDX); secondary ion mass spectrometry (SIMS); time-of-flight SIMS (ToF-SIMS); atom probe imaging or tomography; local electrode atom probe (LEAP) techniques; 3D tomography; or high resolution physical or chemical analysis, to name a few suitable example analytical tools. In particular, such tools can indicate an integrated circuit including at least one semiconductor package including an embedded magnetic inductor.

In some embodiments, the techniques, processes and/or methods described herein can be detected based on the structures formed therefrom. In addition, in some embodiments, the techniques and structures described herein can be detected based on the benefits derived therefrom. Numerous configurations and variations will be apparent in light of this disclosure.

The description may use the phrases “in an embodiment,” “according to some embodiments,” “in accordance with embodiments,” or “in embodiments,” which may each refer to one or more of the same or different embodiments. Furthermore, the terms “comprising,” “including,” “having,” and the like, as used with respect to embodiments of the present disclosure, are synonymous.

The foregoing description and summary are to be understood as being in every respect illustrative and exemplary, but not restrictive, and the scope disclosed herein is not to be determined only from the detailed description of illustrative implementations but according to the full breadth permitted by patent laws. It is to be understood that the implementations shown and described herein are only illustrative of the principles of the present invention and that various modifications may be implemented by those skilled in the art without departing from the scope and spirit.

### Examples

Illustrative examples of the technologies described throughout this disclosure are provided below. Embodiments of these technologies may include any one or more, and any combination of, the examples described below. In some embodiments, at least one of the systems or components set forth in one or more of the preceding figures may be configured as set forth in the following examples.

Example 1 includes a spinal implant assembly comprising: a first endplate having a first non-planar inner surface; a second endplate having a second non-planar inner surface facing toward the first non-planar inner surface; at least one fastener attaching the first endplate and the second endplate to one another; and a layer including a piezoelectric material (piezoelectric layer), the piezoelectric layer between the first non-planar inner surface and the second non-planar inner surface and having a non-planar upper surface facing toward the first non-planar inner surface and a non-planar lower surface facing toward the second non-planar inner surface, wherein, at a front region of the spinal implant assembly, the first non-planar inner surface and the second non-planar inner surface define a vertically biased interface.

Example 2 includes the subject matter of Example 1, wherein: the first endplate has an outer surface defining, at a front region thereof, a first downwardly sloped and horizontally extending nose surface (first nose surface); and the second endplate has an outer surface defining, at a front region thereof, a second downwardly sloped and horizontally extending nose surface (second nose surface), wherein the spinal implant assembly has a downwardly extending nose surface at a front region thereof corresponding to a combination of the first nose surface and the second nose surface.

Example 3 includes the subject matter of Example 1, wherein the first non-planar inner surface has first undulations and the second non-planar inner surface has second undulations, the first undulations and the second undulations defined in a longitudinal direction of the spinal implant assembly and conforming to one another.

Example 4 includes the subject matter of Example 1, wherein the non-planar upper surface of the piezoelectric layer conforms to and is adjacent to the first non-planar inner surface, and the non-planar lower surface of the piezoelectric layer conforms to and is adjacent to the second non-planar inner surface.

Example 5 includes the subject matter of any one of Examples 1-4, wherein the first endplate, the second endplate and the piezoelectric layer are configured such that the first endplate and the second endplate are to cause at least one of a compressive force or a shear force on the piezoelectric layer, the at least one of the compressive force or the shear force to be distributed unevenly throughout the piezoelectric layer.

Example 6 includes the subject matter of any one of Examples 1-5, wherein: the first non-planar inner surface defines, at a front region thereof, a first upwardly sloped surface; and the second non-planar inner surface defines, at a front region thereof, a second upwardly sloped surface, wherein the first upwardly sloped surface and the second upwardly sloped surface conform to and are adjacent to one another.

Example 7 includes the subject matter of Example 6, wherein: a normal vector VT to the first upwardly sloped surface has a horizontal component pointing forward and having a magnitude larger than a magnitude of any vertical component of normal vector VT; and a normal vector VB to the second upwardly sloped surface has a horizontal component pointing backward and having a magnitude larger than a magnitude of any vertical component of normal vector VB.

Example 8 includes the subject matter of any one of Examples 1-7, wherein: the first endplate defines a first lumen therein and a plurality of first holes through sides thereof; the second endplate defines a second lumen therein and a plurality of second holes through sides thereof; and said at least one fastener includes: a plurality of brackets extending within the first lumen and the second lumen, each of the plurality of brackets defining a top hole therein and a bottom hole therein, the top hole in registration with corresponding ones of the plurality of first holes, and the bottom hole in registration with corresponding ones of the plurality of second holes; a plurality of top pins, respective ones of the plurality of top pins extending through the top hole of a corresponding one of said each of the plurality of brackets and through corresponding ones of the plurality of first holes; and a plurality of bottom pins, respective ones of the plurality of bottom pins extending through the bottom hole of a corresponding one of said each of the plurality of brackets and through corresponding ones of the plurality of second holes.

Example 9 includes the subject matter of Example 8, wherein the plurality of brackets include an electrically insulating material.

Example 10 includes the subject matter of Example 9, wherein at least some of the plurality of top pins and the plurality of bottom pins include an electrically conductive material.

Example 11 includes the subject matter of Example 9, wherein the plurality of brackets include an electrically conductive core and cover layers at lateral sides of the electrically conductive core.

Example 12 includes the subject matter of Example 11, wherein the plurality of top pins and the plurality of bottom pins include an electrically insulating material.

Example 13 includes the subject matter of any one of Examples 8-12, wherein individual ones of the plurality of brackets have flat lateral surfaces and rounded top and bottom ends.

Example 14 includes the subject matter of any one of Examples 8-13, wherein: respective ones of the plurality of top pins abut an upper boundary of the top hole of a corresponding one of said each of the plurality of brackets and further define a gap with respect to lower boundaries of corresponding ones of the plurality of first holes; and respective ones of the plurality of bottom pins abut a lower boundary of the bottom hole of a corresponding one of said each of the plurality of brackets and further define a gap with respect to upper boundaries of corresponding ones of the plurality of second holes.

Example 15 includes the subject matter of any one of Examples 1-7, wherein: the first endplate defines a plurality of first holes therein extending vertically; the second endplate defines a plurality of second holes therein extending vertically, respective ones of the plurality of second holes in registration with respective corresponding ones of the plurality of first holes to form respective vertical fastener holes; and said at least one fastener includes a plurality of screws, respective ones of the plurality of screws extending through and engaging threads of respective ones of the vertical fastener holes.

Example 16 includes the subject matter of Example 15, wherein the plurality of screws include lag screws.

Example 17 includes the subject matter of any one of Examples 15-16, wherein the plurality of screws include an electrically insulating material.

Example 18 includes the subject matter of any one of Examples 15-16, wherein the plurality of screws include an electrically conductive material.

Example 19 includes the subject matter of any one of Examples 15-18, wherein the plurality of screws include, at upper surfaces of heads thereof, osseus-friendly lattices.

Example 20 includes the subject matter of Example 19, wherein plurality of screws have a hollow interior, and wherein the osseus-friendly lattices defines apertures that are in communication with the hollow interior.

Example 21 includes the subject matter of any one of Examples 15-20, wherein: the first endplate has a first outer surface including first protrusions thereon, and the second endplate has a second outer surface including second protrusions thereon, the first protrusions and the second protrusions including at least one of teeth or spikes; and the plurality of screws include respective heads at a vertical distance below a topmost portion of the first protrusions.

Example 22 includes the subject matter of any one of Examples 15-21, the plurality of screws including two screws spaced from one another in a longitudinal direction of the spinal implant assembly.

Example 23 includes the subject matter of any one of Examples 15-22, wherein the plurality of second holes are threaded, and the plurality of first holes are defined by smooth boundaries and have a diameter larger than a diameter of the plurality of screws.

Example 24 includes the subject matter of any one of Examples 15-22, wherein the plurality of first holes include only two first holes, and the plurality of second holes include only two second holes, a single one of the two first holes being threaded, and a single one of the two second holes being threaded.

Example 25 includes the subject matter of any one of Examples 15-24, wherein; the plurality of screws include an electrically conductive material; the at least one fastener further includes a plurality of rings having respective convex-conical-frustum-shaped ring surfaces at lower surfaces thereof, and respective concave-conical-frustum-shaped ring surfaces at upper surfaces thereof, the plurality of rings including an electrically insulating material; individual ones of the plurality of screws have convex-conical-frustum-shaped bearing surfaces at undersides of heads thereof, individual ones of the concave-conical-frustum-shaped ring surfaces conforming to and adjacent to respective corresponding ones of the convex-conical-frustum-shaped bearing surfaces of the plurality of screws; at least one of the plurality of first holes or the plurality of second holes define, at entry regions thereof, concave-conical-frustum-shaped ring seat surfaces, individual ones of the concave-conical-frustum-shaped ring seat surfaces conforming to and adjacent to respective corresponding ones of the convex-conical-frustum-shaped ring surfaces; and the plurality of rings form a physical barrier between the heads of the plurality of screws and said at least one of the plurality of first holes or the plurality of second holes.

Example 26 includes the subject matter of any one of Examples 15-24, wherein; the plurality of screws include an electrically conductive material; the at least one fastener further includes a plurality of rings having respective convex collared annulus-shaped ring surfaces at lower surfaces thereof, and respective concave collared annulus-shaped ring surfaces at upper surfaces thereof, the plurality of rings including an electrically insulating material; individual ones of the plurality of screws have flat bearing surfaces at undersides of heads thereof, individual ones of the concave collared annulus-shaped ring surfaces conforming to and adjacent to respective corresponding heads of the plurality of screws; at least one of the plurality of first holes or the plurality of second holes define, at entry regions thereof, concave collared annulus-shaped ring seat surfaces, individual ones of the ring seat surfaces conforming to and adjacent to respective corresponding ones of the convex collared annulus-shaped ring surfaces; and the plurality of rings form a physical barrier between the heads of the plurality of screws and said at least one of the plurality of first holes or the plurality of second holes.

Example 27 includes the subject matter of Example 1-7, wherein: the first endplate defines a first lumen therein and a plurality of first holes through sides thereof; and said at least one fastener includes: a plurality of struts extending from the second non-planar inner surface and within the first lumen, each of the plurality of struts defining a strut hole therein that is in registration with corresponding ones of the plurality of first holes; and a plurality of pins, respective ones of the plurality of pins extending through the strut hole of a corresponding one of said each of the plurality of struts and through corresponding ones of the plurality of first holes.

Example 28 includes the subject matter of Example 27, wherein the plurality of struts are unitary with the second endplate.

Example 29 includes the subject matter of any one of Examples 27-28, wherein the plurality of struts have flat lateral surfaces and rounded top edges.

Example 30 includes the subject matter of any one of Examples 27-29, further including a pair of electrically insulating washers flanking lateral surfaces of individual ones of the plurality of struts and electrically insulating the plurality of struts from the first endplate.

Example 31 includes the subject matter of any one of Examples 27-30, wherein the plurality of pins include an electrically insulating material.

Example 32 includes a kit for a spinal implant assembly comprising: a first endplate having a first non-planar inner surface; a second endplate having a second non-planar inner surface; at least one fastener to attach the first endplate and the second endplate to one another; and a layer including a piezoelectric material (piezoelectric layer), wherein the piezoelectric layer is configured to have, in the spinal implant assembly, a non-planar upper surface facing toward the first non-planar inner surface and a non-planar lower surface facing toward the second non-planar inner surface, and wherein the first non-planar inner surface and the second non-planar inner surface are configured to define, in the spinal implant assembly, a vertically biased interface.

Example 33 includes the subject matter of Example 32, wherein: the first endplate has an outer surface defining, at a front region thereof, a first downwardly sloped and horizontally extending nose surface (first nose surface); and the second endplate has an outer surface defining, at a front region thereof, a second downwardly sloped and horizontally extending nose surface (second nose surface), the first endplate and the second endplate being configured such that the spinal implant assembly has a downwardly extending nose surface at a front region thereof corresponding to a combination of the first nose surface and the second nose surface.

Example 34 includes the subject matter of any one of Examples 32-33, wherein the first non-planar inner surface has first undulations and the second non-planar inner surface has second undulations, the first undulations and the second undulations defined in a longitudinal direction of the spinal implant assembly and conforming to one another.

Example 35 includes the subject matter of Example 34, wherein the piezoelectric layer is flat prior to incorporation into the spinal implant assembly, and, in the spinal implant assembly, the non-planar upper surface of the piezoelectric layer conforms to and is adjacent to the first non-planar inner surface, and the non-planar lower surface of the piezoelectric layer conforms to and is adjacent to the second non-planar inner surface.

Example 36 includes the subject matter of any one of Examples 33-35, wherein the first endplate, the second endplate and the piezoelectric layer are configured such that the first endplate and the second endplate are to cause at least one of a compressive force or a shear force on the piezoelectric layer, the at least one of the compressive force or the shear force to be distributed unevenly throughout the piezoelectric layer.

Example 37 includes the subject matter of any one of Examples 33-36, wherein: the first non-planar inner surface defines, at a front region thereof, a first upwardly sloped surface; and the second non-planar inner surface defines, at a front region thereof, a second upwardly sloped surface, wherein the first upwardly sloped surface and the second upwardly sloped surface conform to one another.

Example 38 includes the subject matter of Example 37, wherein: a normal vector VT to the first upwardly sloped surface has a horizontal component pointing forward and having a magnitude larger than a magnitude of any vertical component of normal vector VT; and a normal vector VB to the second upwardly sloped surface has a horizontal component pointing backward and having a magnitude larger than a magnitude of any vertical component of normal vector VB.

Example 39 includes the subject matter of any one of Examples 33-38, wherein: the first endplate defines a first lumen therein and a plurality of first holes through sides thereof; the second endplate defines a second lumen therein and a plurality of second holes through sides thereof; and said at least one fastener includes: a plurality of brackets to extend within the first lumen and the second lumen, each of the plurality of brackets defining a top hole therein and a bottom hole therein, the top hole to be in registration with corresponding ones of the plurality of first holes, and the bottom hole to be in registration with corresponding ones of the plurality of second holes; a plurality of top pins, respective ones of the plurality of top pins to extend through the top hole of a corresponding one of said each of the plurality of brackets and through corresponding ones of the plurality of first holes; and a plurality of bottom pins, respective ones of the plurality of bottom pins to extend through the bottom hole of a corresponding one of said each of the plurality of brackets and through corresponding ones of the plurality of second holes.

Example 40 includes the subject matter of Example 39, wherein the plurality of brackets include an electrically insulating material.

Example 41 includes the subject matter of Example 40, wherein at least some of the plurality of top pins and the plurality of bottom pins include an electrically conductive material.

Example 42 includes the subject matter of Example 40, wherein the plurality of brackets include an electrically conductive core and cover layers at lateral sides of the electrically conductive core.

Example 43 includes the subject matter of Example 42, wherein the plurality of top pins and the plurality of bottom pins include an electrically insulating material.

Example 44 includes the subject matter of any one of Examples 39-43, wherein individual ones of the plurality of brackets have flat lateral surfaces and rounded top and bottom ends.

Example 45 includes the subject matter of any one of Examples 39-44, wherein: respective ones of the plurality of top pins are to abut an upper boundary of the top hole of a corresponding one of said each of the plurality of brackets and to further define a gap with respect to lower boundaries of corresponding ones of the plurality of first holes; and respective ones of the plurality of bottom pins are to abut a lower boundary of the bottom hole of a corresponding one of said each of the plurality of brackets and to further define a gap with respect to upper boundaries of corresponding ones of the plurality of second holes.

Example 46 includes the subject matter of any one of Examples 33-38, wherein: the first endplate defines a plurality of first holes therein extending vertically; the second endplate defines a plurality of second holes therein extending vertically, respective ones of the plurality of second holes to be in registration with respective corresponding ones of the plurality of first holes to form respective vertical fastener holes; and said at least one fastener includes a plurality of screws, respective ones of the plurality of screws to extend through and to engage threads of respective ones of the vertical fastener holes.

Example 47 includes the subject matter of Example 46, wherein the plurality of screws include lag screws.

Example 48 includes the subject matter of any one of Examples 46-47, wherein the plurality of screws include an electrically insulating material.

Example 49 includes the subject matter of any one of Examples 46-47, wherein the plurality of screws include an electrically conductive material.

Example 50 includes the subject matter of any one of Examples 46-49, wherein the plurality of screws include, at upper surfaces of heads thereof, osseus-friendly lattices.

Example 51 includes the subject matter of Example 50, wherein plurality of screws have a hollow interior, and wherein the osseus-friendly lattices defines apertures that are in communication with the hollow interior.

Example 52 includes the subject matter of any one of Examples 46-51, wherein: the first endplate has a first outer surface including first protrusions thereon, and the second endplate has a second outer surface including second protrusions thereon, the first protrusions and the second protrusions including at least one of teeth or spikes; and the plurality of screws include respective heads to be, in the spinal implant assembly, at a vertical distance below a topmost portion of the first protrusions.

Example 53 includes the subject matter of any one of Examples 46-52, the plurality of screws including only two screws.

Example 54 includes the subject matter of any one of Examples 46-53, wherein the plurality of second holes are threaded, and the plurality of first holes are defined by smooth boundaries and have a diameter larger than a diameter of the plurality of screws.

Example 55 includes the subject matter of any one of Examples 46-53, wherein the plurality of first holes include only two first holes, and the plurality of second holes include only two second holes, a single one of the two first holes being threaded, and a single one of the two second holes being threaded.

Example 56 includes the subject matter of any one of Examples 46-55, wherein; the plurality of screws include an electrically conductive material; the at least one fastener further includes a plurality of rings having respective convex-conical-frustum-shaped ring surfaces at lower surfaces thereof, and respective concave-conical-frustum-shaped ring surfaces at upper surfaces thereof, the plurality of rings including an electrically insulating material; individual ones of the plurality of screws have convex-conical-frustum-shaped bearing surfaces at undersides of heads thereof, individual ones of the concave-conical-frustum-shaped ring surfaces conforming to respective corresponding ones of the convex-conical-frustum-shaped bearing surfaces of the plurality of screws; at least one of the plurality of first holes or the plurality of second holes define, at entry regions thereof, concave-conical-frustum-shaped ring seat surfaces, individual ones of the concave-conical-frustum-shaped ring seat surfaces conforming to respective corresponding ones of the convex-conical-frustum-shaped ring surfaces; and the plurality of rings are to form a physical barrier between the heads of the plurality of screws and said at least one of the plurality of first holes or the plurality of second holes.

Example 57 includes the subject matter of any one of Examples 46-55, wherein; the plurality of screws include an electrically conductive material; the at least one fastener further includes a plurality of rings having respective convex collared annulus-shaped ring surfaces at lower surfaces thereof, and respective concave collared annulus-shaped ring surfaces at upper surfaces thereof, the plurality of rings including an electrically insulating material; individual ones of the plurality of screws have flat bearing surfaces at undersides of heads thereof, individual ones of the concave collared annulus-shaped ring surfaces conforming to respective corresponding heads of the plurality of screws; at least one of the plurality of first holes or the plurality of second holes define, at entry regions thereof, concave collared annulus-shaped ring seat surfaces, individual ones of the ring seat surfaces conforming to respective corresponding ones of the convex collared annulus-shaped ring surfaces; and the plurality of rings are to form a physical barrier between the heads of the plurality of screws and said at least one of the plurality of first holes or the plurality of second holes.

Example 58 includes the subject matter of Example 44, wherein: the first endplate defines a first lumen therein and a plurality of first holes through sides thereof; and said at least one fastener includes: a plurality of struts extending from the second non-planar inner surface and within the first lumen, each of the plurality of struts defining a strut hole therein that is to be in registration with corresponding ones of the plurality of first holes; and a plurality of pins, respective ones of the plurality of pins to extend through the strut hole of a corresponding one of said each of the plurality of struts and through corresponding ones of the plurality of first holes.

Example 59 includes the subject matter of Example 58, wherein the plurality of struts are unitary with the second endplate.

Example 60 includes the subject matter of any one of Examples 58-59, wherein the plurality of struts have flat lateral surfaces and rounded top edges.

Example 61 includes the subject matter of any one of Examples 58-60, further including a pair of electrically insulating washers to flank lateral surfaces of individual ones of the plurality of struts and to electrically insulate the plurality of struts from the first endplate.

Example 62 includes the subject matter of any one of Examples 58-61, wherein the plurality of pins include an electrically insulating material.

Example 63 includes a method to make a kit for a spinal implant assembly, the method comprising: providing a first endplate having a first non-planar inner surface; providing a second endplate having a second non-planar inner surface; providing at least one fastener to attach the first endplate and the second endplate to one another; and providing a layer including a piezoelectric material (piezoelectric layer), wherein the piezoelectric layer is configured to have, in the spinal implant assembly, a non-planar upper surface facing toward the first non-planar inner surface and a non-planar lower surface facing toward the second non-planar inner surface, and wherein the first non-planar inner surface and the second non-planar inner surface are configured to define, in the spinal implant assembly, a vertically biased interface.

Example 64 includes the subject matter of Example 63, wherein at least one of providing the first endplate, providing the second endplate, and providing the at least one fastener include using three-dimensional (3D) printing.

What is claimed is:

1. A spinal implant assembly comprising:
a first endplate having a first non-planar inner surface;
a second endplate having a second non-planar inner surface facing toward the first non-planar inner surface;
at least one fastener attaching the first endplate and the second endplate to one another; and
a layer including a piezoelectric material (piezoelectric layer), the piezoelectric layer between the first non-planar inner surface and the second non-planar inner surface and having a non-planar upper surface facing toward the first non-planar inner surface and a non-planar lower surface facing toward the second non-planar inner surface, wherein, at a front region of the spinal implant assembly, the first non-planar inner surface and the second non-planar inner surface define a vertically biased interface.

2. The spinal implant assembly of claim 1, wherein:
the first endplate has an outer surface defining, at a front region thereof, a first downwardly sloped and horizontally extending nose surface (first nose surface); and
the second endplate has an outer surface defining, at a front region thereof, a second downwardly sloped and horizontally extending nose surface (second nose surface), wherein the spinal implant assembly has a downwardly extending nose surface at a front region thereof corresponding to a combination of the first nose surface and the second nose surface.

3. The spinal implant assembly of claim 1, wherein the first non-planar inner surface has first undulations and the second non-planar inner surface has second undulations, the first undulations and the second undulations defined in a longitudinal direction of the spinal implant assembly and conforming to one another.

4. The spinal implant assembly of claim 1, wherein the non-planar upper surface of the piezoelectric layer conforms to and is adjacent to the first non-planar inner surface, and the non-planar lower surface of the piezoelectric layer conforms to and is adjacent to the second non-planar inner surface.

5. The spinal implant assembly of claim 1, wherein the first endplate, the second endplate and the piezoelectric layer are configured such that the first endplate and the second endplate are to cause at least one of a compressive force or a shear force on the piezoelectric layer, the at least one of the compressive force or the shear force to be distributed unevenly throughout the piezoelectric layer.

6. The spinal implant assembly of claim 1, wherein:
the first non-planar inner surface defines, at a front region thereof, a first upwardly sloped surface; and
the second non-planar inner surface defines, at a front region thereof, a second upwardly sloped surface, wherein the first upwardly sloped surface and the second upwardly sloped surface conform to and are adjacent to one another.

7. The spinal implant assembly of claim 6, wherein:
a normal vector V_{T} to the first upwardly sloped surface has a horizontal component pointing forward and having a magnitude larger than a magnitude of any vertical component of normal vector V_{T}; and
a normal vector V_{B} to the second upwardly sloped surface has a horizontal component pointing backward and having a magnitude larger than a magnitude of any vertical component of normal vector V_{B}.

8. The spinal implant assembly of claim 1, wherein:
the first endplate defines a first lumen therein and a plurality of first holes through sides thereof;
the second endplate defines a second lumen therein and a plurality of second holes through sides thereof; and
said at least one fastener includes:
a plurality of brackets extending within the first lumen and the second lumen, each of the plurality of brackets defining a top hole therein and a bottom hole therein, the top hole in registration with corresponding ones of the plurality of first holes, and the bottom hole in registration with corresponding ones of the plurality of second holes;
a plurality of top pins, respective ones of the plurality of top pins extending through the top hole of a corresponding one of said each of the plurality of brackets and through corresponding ones of the plurality of first holes; and
a plurality of bottom pins, respective ones of the plurality of bottom pins extending through the bottom hole of a corresponding one of said each of the plurality of brackets and through corresponding ones of the plurality of second holes.

9. The spinal implant assembly of claim 8, wherein the plurality of brackets include an electrically insulating material.

10. The spinal implant assembly of claim 9, wherein at least some of the plurality of top pins and the plurality of bottom pins include an electrically conductive material.

11. The spinal implant assembly of claim 9, wherein the plurality of brackets include an electrically conductive core and cover layers at lateral sides of the electrically conductive core.

12. The spinal implant assembly of claim 11, wherein the plurality of top pins and the plurality of bottom pins include an electrically insulating material.

13. The spinal implant assembly of claim 8, wherein individual ones of the plurality of brackets have flat lateral surfaces and rounded top and bottom ends.

14. The spinal implant assembly of claim 8, wherein:
respective ones of the plurality of top pins abut an upper boundary of the top hole of a corresponding one of said each of the plurality of brackets and further define a gap with respect to lower boundaries of corresponding ones of the plurality of first holes; and
respective ones of the plurality of bottom pins abut a lower boundary of the bottom hole of a corresponding one of said each of the plurality of brackets and further define a gap with respect to upper boundaries of corresponding ones of the plurality of second holes.

15. The spinal implant assembly of claim 1, wherein:
the first endplate defines a plurality of first holes therein extending vertically;
the second endplate defines a plurality of second holes therein extending vertically, respective ones of the plurality of second holes in registration with respective corresponding ones of the plurality of first holes to form respective vertical fastener holes; and
said at least one fastener includes a plurality of screws, respective ones of the plurality of screws extending through and engaging threads of respective ones of the vertical fastener holes.

16. The spinal implant assembly of claim 15, wherein the plurality of screws include lag screws.

17. The spinal implant assembly of claim 15, wherein the plurality of screws include an electrically insulating material.

18. The spinal implant assembly of claim 15, wherein the plurality of screws include an electrically conductive material.

19. The spinal implant assembly of claim 15, wherein the plurality of screws include, at upper surfaces of heads thereof, osseus-friendly lattices.

20. The spinal implant assembly of claim 19, wherein plurality of screws have a hollow interior, and wherein the osseus-friendly lattices defines apertures that are in communication with the hollow interior.

21. The spinal implant assembly of claim 15, wherein:
the first endplate has a first outer surface including first protrusions thereon, and the second endplate has a second outer surface including second protrusions thereon, the first protrusions and the second protrusions including at least one of teeth or spikes; and
the plurality of screws include respective heads at a vertical distance below a topmost portion of the first protrusions.

22. The spinal implant assembly of claim 15, the plurality of screws including two screws spaced from one another in a longitudinal direction of the spinal implant assembly.

23. The spinal implant assembly of claim 15, wherein the plurality of second holes are threaded, and the plurality of first holes are defined by smooth boundaries and have a diameter larger than a diameter of the plurality of screws.

24. The spinal implant assembly of claim 15, wherein the plurality of first holes include only two first holes, and the plurality of second holes include only two second holes, a single one of the two first holes being threaded, and a single one of the two second holes being threaded.

25. The spinal implant assembly of claim 15, wherein;
the plurality of screws include an electrically conductive material;
the at least one fastener further includes a plurality of rings having respective convex-conical-frustum-shaped ring surfaces at lower surfaces thereof, and respective concave-conical-frustum-shaped ring surfaces at upper surfaces thereof, the plurality of rings including an electrically insulating material;
individual ones of the plurality of screws have convex-conical-frustum-shaped bearing surfaces at undersides of heads thereof, individual ones of the concave-conical-frustum-shaped ring surfaces conforming to and adjacent to respective corresponding ones of the convex-conical-frustum-shaped bearing surfaces of the plurality of screws;
at least one of the plurality of first holes or the plurality of second holes define, at entry regions thereof, concave-conical-frustum-shaped ring seat surfaces, individual ones of the concave-conical-frustum-shaped ring seat surfaces conforming to and adjacent to respective corresponding ones of the convex-conical-frustum-shaped ring surfaces; and
the plurality of rings form a physical barrier between the heads of the plurality of screws and said at least one of the plurality of first holes or the plurality of second holes.

26. The spinal implant assembly of claim 15, wherein;
the plurality of screws include an electrically conductive material;
the at least one fastener further includes a plurality of rings having respective convex collared annulus-shaped ring surfaces at lower surfaces thereof, and respective concave collared annulus-shaped ring surfaces at upper surfaces thereof, the plurality of rings including an electrically insulating material;
individual ones of the plurality of screws have flat bearing surfaces at undersides of heads thereof, individual ones of the concave collared annulus-shaped ring surfaces conforming to and adjacent to respective corresponding heads of the plurality of screws;
at least one of the plurality of first holes or the plurality of second holes define, at entry regions thereof, concave collared annulus-shaped ring seat surfaces, individual ones of the ring seat surfaces conforming to and adjacent to respective corresponding ones of the convex collared annulus-shaped ring surfaces; and
the plurality of rings form a physical barrier between the heads of the plurality of screws and said at least one of the plurality of first holes or the plurality of second holes.

27. The spinal implant assembly of claim 1, wherein:
the first endplate defines a first lumen therein and a plurality of first holes through sides thereof; and
said at least one fastener includes:
a plurality of struts extending from the second non-planar inner surface and within the first lumen, each of the plurality of struts defining a strut hole therein that is in registration with corresponding ones of the plurality of first holes; and
a plurality of pins, respective ones of the plurality of pins extending through the strut hole of a corresponding one of said each of the plurality of struts and through corresponding ones of the plurality of first holes.

28. The spinal implant assembly of claim 27, wherein the plurality of struts are unitary with the second endplate.

29. The spinal implant assembly of claim 27, wherein the plurality of struts have flat lateral surfaces and rounded top edges.

30. The spinal implant assembly of claim 27, further including a pair of electrically insulating washers flanking lateral surfaces of individual ones of the plurality of struts and electrically insulating the plurality of struts from the first endplate.

31. The spinal implant assembly of claim 27, wherein the plurality of pins include an electrically insulating material.

32. A kit for a spinal implant assembly comprising:
a first endplate having a first non-planar inner surface;
a second endplate having a second non-planar inner surface;
at least one fastener to attach the first endplate and the second endplate to one another; and
a layer including a piezoelectric material (piezoelectric layer), wherein the piezoelectric layer is configured to have, in the spinal implant assembly, a non-planar upper surface facing toward the first non-planar inner surface and a non-planar lower surface facing toward the second non-planar inner surface, and wherein the first non-planar inner surface and the second non-planar inner surface are configured to define, in the spinal implant assembly, a vertically biased interface.

33. The kit of claim 32, wherein:
the first endplate has an outer surface defining, at a front region thereof, a first downwardly sloped and horizontally extending nose surface (first nose surface); and
the second endplate has an outer surface defining, at a front region thereof, a second downwardly sloped and horizontally extending nose surface (second nose surface), the first endplate and the second endplate being configured such that the spinal implant assembly has a downwardly extending nose surface at a front region thereof corresponding to a combination of the first nose surface and the second nose surface.

34. The kit of claim 32, wherein the first non-planar inner surface has first undulations and the second non-planar inner surface has second undulations, the first undulations and the second undulations defined in a longitudinal direction of the spinal implant assembly and conforming to one another.

35. The kit of claim 34, wherein the piezoelectric layer is flat prior to incorporation into the spinal implant assembly, and, in the spinal implant assembly, the non-planar upper surface of the piezoelectric layer conforms to and is adjacent to the first non-planar inner surface, and the non-planar lower surface of the piezoelectric layer conforms to and is adjacent to the second non-planar inner surface.

36. The kit of claim 33, wherein the first endplate, the second endplate and the piezoelectric layer are configured such that the first endplate and the second endplate are to cause at least one of a compressive force or a shear force on the piezoelectric layer, the at least one of the compressive force or the shear force to be distributed unevenly throughout the piezoelectric layer.

37. The kit of claim 33, wherein:
the first non-planar inner surface defines, at a front region thereof, a first upwardly sloped surface; and
the second non-planar inner surface defines, at a front region thereof, a second upwardly sloped surface, wherein the first upwardly sloped surface and the second upwardly sloped surface conform to one another.

38. The kit of claim 37, wherein:
a normal vector V_{T} to the first upwardly sloped surface has a horizontal component pointing forward and having a magnitude larger than a magnitude of any vertical component of normal vector V_{T}; and
a normal vector V_{B} to the second upwardly sloped surface has a horizontal component pointing backward and having a magnitude larger than a magnitude of any vertical component of normal vector V_{B}.

39. The kit of claim 33, wherein:
the first endplate defines a first lumen therein and a plurality of first holes through sides thereof;
the second endplate defines a second lumen therein and a plurality of second holes through sides thereof; and
said at least one fastener includes:
a plurality of brackets to extend within the first lumen and the second lumen, each of the plurality of brackets defining a top hole therein and a bottom hole therein, the top hole to be in registration with corresponding ones of the plurality of first holes, and the bottom hole to be in registration with corresponding ones of the plurality of second holes;
a plurality of top pins, respective ones of the plurality of top pins to extend through the top hole of a corresponding one of said each of the plurality of brackets and through corresponding ones of the plurality of first holes; and
a plurality of bottom pins, respective ones of the plurality of bottom pins to extend through the bottom hole of a corresponding one of said each of the plurality of brackets and through corresponding ones of the plurality of second holes.

40. The kit of claim 39, wherein the plurality of brackets include an electrically insulating material.

41. The kit of claim 40, wherein at least some of the plurality of top pins and the plurality of bottom pins include an electrically conductive material.

42. The kit of claim 40, wherein the plurality of brackets include an electrically conductive core and cover layers at lateral sides of the electrically conductive core.

43. The kit of claim 42, wherein the plurality of top pins and the plurality of bottom pins include an electrically insulating material.

44. The kit of claim 39, wherein individual ones of the plurality of brackets have flat lateral surfaces and rounded top and bottom ends.

45. The kit of claim 39, wherein:
respective ones of the plurality of top pins are to abut an upper boundary of the top hole of a corresponding one of said each of the plurality of brackets and to further define a gap with respect to lower boundaries of corresponding ones of the plurality of first holes; and
respective ones of the plurality of bottom pins are to abut a lower boundary of the bottom hole of a corresponding one of said each of the plurality of brackets and to further define a gap with respect to upper boundaries of corresponding ones of the plurality of second holes.

46. The kit of claim 33, wherein:
the first endplate defines a plurality of first holes therein extending vertically;
the second endplate defines a plurality of second holes therein extending vertically, respective ones of the plurality of second holes to be in registration with respective corresponding ones of the plurality of first holes to form respective vertical fastener holes; and
said at least one fastener includes a plurality of screws, respective ones of the plurality of screws to extend through and to engage threads of respective ones of the vertical fastener holes.

47. The kit of claim 46, wherein the plurality of screws include lag screws.

48. The kit of claim 46, wherein the plurality of screws include an electrically insulating material.

49. The kit of claim 46, wherein the plurality of screws include an electrically conductive material.

50. The kit of claim 46, wherein the plurality of screws include, at upper surfaces of heads thereof, osseus-friendly lattices.

51. The kit of claim 50, wherein plurality of screws have a hollow interior, and wherein the osseus-friendly lattices defines apertures that are in communication with the hollow interior.

52. The kit of claim 46, wherein:
the first endplate has a first outer surface including first protrusions thereon, and the second endplate has a second outer surface including second protrusions thereon, the first protrusions and the second protrusions including at least one of teeth or spikes; and
the plurality of screws include respective heads to be, in the spinal implant assembly, at a vertical distance below a topmost portion of the first protrusions.

53. The kit of claim 46, the plurality of screws including only two screws.

54. The kit of claim 46, wherein the plurality of second holes are threaded, and the plurality of first holes are defined by smooth boundaries and have a diameter larger than a diameter of the plurality of screws.

55. The kit of claim 46, wherein the plurality of first holes include only two first holes, and the plurality of second holes include only two second holes, a single one of the two first holes being threaded, and a single one of the two second holes being threaded.

56. The kit of claim 46, wherein;
the plurality of screws include an electrically conductive material;
the at least one fastener further includes a plurality of rings having respective convex-conical-frustum-shaped ring surfaces at lower surfaces thereof, and respective concave-conical-frustum-shaped ring surfaces at upper surfaces thereof, the plurality of rings including an electrically insulating material;
individual ones of the plurality of screws have convex-conical-frustum-shaped bearing surfaces at undersides of heads thereof, individual ones of the concave-conical-frustum-shaped ring surfaces conforming to respective corresponding ones of the convex-conical-frustum-shaped bearing surfaces of the plurality of screws;
at least one of the plurality of first holes or the plurality of second holes define, at entry regions thereof, concave-conical-frustum-shaped ring seat surfaces, individual ones of the concave-conical-frustum-shaped ring seat surfaces conforming to respective corresponding ones of the convex-conical-frustum-shaped ring surfaces; and
the plurality of rings are to form a physical barrier between the heads of the plurality of screws and said at least one of the plurality of first holes or the plurality of second holes.

57. The kit of claim 46, wherein;
the plurality of screws include an electrically conductive material;
the at least one fastener further includes a plurality of rings having respective convex collared annulus-shaped ring surfaces at lower surfaces thereof, and respective concave collared annulus-shaped ring surfaces at upper surfaces thereof, the plurality of rings including an electrically insulating material;
individual ones of the plurality of screws have flat bearing surfaces at undersides of heads thereof, individual ones of the concave collared annulus-shaped ring surfaces conforming to respective corresponding heads of the plurality of screws;
at least one of the plurality of first holes or the plurality of second holes define, at entry regions thereof, concave collared annulus-shaped ring seat surfaces, individual ones of the ring seat surfaces conforming to respective corresponding ones of the convex collared annulus-shaped ring surfaces; and
the plurality of rings are to form a physical barrier between the heads of the plurality of screws and said at least one of the plurality of first holes or the plurality of second holes.

58. The kit of claim 44, wherein:
the first endplate defines a first lumen therein and a plurality of first holes through sides thereof; and
said at least one fastener includes:
a plurality of struts extending from the second non-planar inner surface and within the first lumen, each of the plurality of struts defining a strut hole therein that is to be in registration with corresponding ones of the plurality of first holes; and
a plurality of pins, respective ones of the plurality of pins to extend through the strut hole of a corresponding one of said each of the plurality of struts and through corresponding ones of the plurality of first holes.

59. The kit of claim 58, wherein the plurality of struts are unitary with the second endplate.

60. The kit of claim 58, wherein the plurality of struts have flat lateral surfaces and rounded top edges.

61. The kit of claim 58, further including a pair of electrically insulating washers to flank lateral surfaces of individual ones of the plurality of struts and to electrically insulate the plurality of struts from the first endplate.

62. The kit of claim 58, wherein the plurality of pins include an electrically insulating material.

63. A method to make a kit for a spinal implant assembly, the method comprising:
providing a first endplate having a first non-planar inner surface;
providing a second endplate having a second non-planar inner surface;
providing at least one fastener to attach the first endplate and the second endplate to one another; and
providing a layer including a piezoelectric material (piezoelectric layer), wherein the piezoelectric layer is configured to have, in the spinal implant assembly, a non-planar upper surface facing toward the first non-planar inner surface and a non-planar lower surface facing toward the second non-planar inner surface, and wherein the first non-planar inner surface and the second non-planar inner surface are configured to define, in the spinal implant assembly, a vertically biased interface.

64. The method of claim 63, wherein at least one of providing the first endplate, providing the second endplate, and providing the at least one fastener include using three-dimensional (3D) printing.
